(19) United States
(12) Patent Application Publication
(10) Pub. No.: US 2022/0112531 A1
(43) Pub. Date: Apr. 14, 2022

(54) HOST YEAST CELLS AND METHODS USEFUL FOR PRODUCING INDIGOIDINE

(71) Applicants: THE REGENTS OF THE UNIVERSITY OF CALIFORNIA, Oakland, CA (US); NATIONAL TECHNOLOGY AND ENGINEERING SOLUTIONS OF SANDIA, LLC, Albuquerque, NM (US)

(72) Inventors: Maren Wehrs, Emeryville, CA (US); John M. Gladden, Alameda, CA (US); Aindrila Mukhopadhyay, Oakland, CA (US)

(21) Appl. No.: 17/405,927

(22) Filed: Aug. 18, 2021

Related U.S. Application Data
(63) Continuation of application No. PCT/US20/19079, filed on Feb. 20, 2020.
(60) Provisional application No. 62/807,969, filed on Feb. 20, 2019.
(60) Provisional application No. 62/961,351, filed on Jan. 15, 2020.

(51) Int. Cl. C12P 17/16; C12N 15/81

(52) U.S. Cl. CPC C12P 17/165; C12R 2001/865; C12N 15/81

(57) ABSTRACT

The present invention provides for a genetically modified fungal host cell capable of producing indigoidine, wherein the host cell comprises a non-ribosomal peptide synthetase (NRPS) that converts glutamine to indigoidine.

### CROSS-REFERENCE TO RELATED APPLICATIONS

The application claims priority as a continuation application to International PCT Patent Application No. PCT/US2020/019079, filed Feb. 20, 2020, which claims priority to U.S. Provisional Patent Application Ser. Nos. 62/807,969, filed Feb. 20, 2019, and 62/961,351, filed Jan. 15, 2020, all of which are herein incorporated by reference in their entireties.

### STATEMENT OF GOVERNMENTAL SUPPORT

The invention was made with government support under Contract Nos. DE-AC02-05CH11231 awarded by the U.S. Department of Energy. The government has certain rights in the invention.

### FIELD OF THE INVENTION

The present invention is in the field of production of indigoidine.

### BACKGROUND OF THE INVENTION

Microbial metabolic pathway discovery and engineering efforts have led to an increasing number of biotechnological processes in diverse sectors of our economy, ranging from energy to health and medicine, as well as food and agriculture. Industrial-scale microbial production environments are profoundly different from the cultivation environments commonly used at lab scale. Thus, beyond pathway engineering, understanding microbial physiology in these different environments is essential to translate proof-of-concept bioprocesses from shake flasks to industrially-relevant bioreactor setups. During large-scale biotechnological production processes, insufficient mixing commonly leads to micro-environmental inhomogeneities with severe concentration gradients of important cultivation characteristics, particularly dissolved oxygen and carbon sources. The benefit of using facultative anaerobic microbes in industrial processes arises from their ability to switch between fermentative and respiratory metabolism to produce ATP depending on the availability of oxygen without loss of viability. However, fluctuations in dissolved oxygen and carbon sources are recognized to trigger metabolic and transcriptional responses, with unfavorable effects on productivity.

Public awareness of eco-safety and health concerns associated with the production and use of certain toxic compounds used throughout the commercial sector has given rise to a growing demand for environmentally sustainable natural products. Secondary metabolites such as polyketides and non-ribosomal peptides (NRPs) have generated significant interest because of their important roles in a wide range of industries including pharmaceuticals, polymers, flavors and fragrances, and natural dyes. Natural dyes have been used for centuries to color textiles, cosmetics, and food ingredients. Current industrial production of dyes is predominantly achieved via chemical synthesis, which can involve toxic precursors such as petroleum-derived aniline and generate hazardous chemicals as byproducts of the process. Microbial production of these dyes has the potential to address the environmental concerns of harsh chemical synthesis while providing higher production levels and purity than achieved by native sources.

Indigoidine is a natural blue pigment natively produced by several bacteria via biosynthetic gene clusters. This 3′,3′-bipyridyl pigment is formed through condensation of two molecules of L-glutamine catalyzed by a non-ribosomal peptide synthetase (NRPS). NRPS are large assembly-line enzymes and are organized in modules that are each responsible for the introduction of one L-amino acid to the NRP. Each module consists of several domains with defined functions that synthesize NRPs in a sequential multi-step process. The secondary metabolite class of NRPs includes molecules with a range of pharmaceutical applications, such as immunosuppressants, antibiotics, anticancer drugs and antiviral compounds. However, the low NRP production levels from native hosts and their complex chemical structures impede mass production by purification from biological material or chemical synthesis. Furthermore, despite the availability of biosynthetic tools for metabolic engineering and pathway discovery, optimization of NRP production in their natural hosts remains challenging.

Saccharomyces cerevisiae is not only used extensively for proof-of-concept pathway studies but also as a host for many applied industrial processes. In contrast to many other fungal or bacterial hosts, S. cerevisiae adjusts its energy metabolism based on the nature of available carbon sources via carbon catabolite repression. Even under aerobic conditions, S. cerevisiae predominantly metabolizes glucose by fermentation leading to the production of ethanol, glycerol and carbon dioxide (FIG. 1A, red arrows). Upon glucose depletion, the non-fermentable products of fermentation ethanol and glycerol can serve as carbon sources, requiring a shift to respiratory mode. The metabolic shift from fermentative to respiratory growth is accompanied by changes of carbon flux and gene expression throughout the whole central metabolism. Under purely fermentative conditions, a redirection of metabolic flux from the tricarboxylic acid (TCA) cycle towards fermentative pathways results in a low activity of the TCA cycle. When switching from fermentative to respiratory conditions, the flux to the TCA cycle increases significantly to enable respiration (FIG. 1A, blue arrows). Thus, activity of the TCA cycle presents an appropriate proxy to distinguish metabolic states in S. cerevisiae. While the effect of the metabolic state on native pathways and products has been investigated, its effect on engineered pathways and biosynthetic products remains understudied.

The basidiomycete Rhodosporidium toruloides, also known as Rhodotorula toruloides, is emerging as a robust and metabolically flexible host for bioproduction. This oleaginous red yeast natively produces high amounts of lipids, carotenoids and industrially relevant enzymes. Furthermore, R. toruloides features many host characteristics vital for commercial-scale production, such as the capacity to grow to high cell densities, the ability to utilize a wide range of nitrogen and carbon sources and tolerance to inhibitory compounds found in unrefined substrates. In addition to its native and convenient industrial features, recent development in metabolic engineering tools and -omics techniques for R. toruloides enabled not only further optimization of native production levels but also the expansion of target molecules produced in this host. While R. toruloides has successfully been engineered to produce heterologous products from pathways that natively have high carbon flux, like fatty acid-derived products and non-native terpenes, R. toruloides has not been explored for the production heterologous NRPs.

### SUMMARY OF THE INVENTION

The present invention provides for a genetically modified fungal host cell capable of producing indigoidine, wherein the host cell comprises a non-ribosomal peptide synthetase (NRPS) that converts glutamine to indigoidine.

In some embodiments, the fungal host cell is a yeast host cell. In some embodiments, the yeast host cell is a non-oleaginous yeast. In some embodiments, the yeast host cell is a non-oleaginous yeast. In some embodiments, the non-oleaginous yeast is a Saccharomyces species. In some embodiments, the Saccharomyces species is Saccharomyces cerevisiae. In some embodiments, the yeast host cell is an oleaginous yeast. In some embodiments, the oleaginous yeast is a Rhodosporidium species. In some embodiments, the Rhodosporidium species is Rhodosporidium toruloides.

In some embodiments, the NRPS is heterologous to the host cell. In some embodiments, the NRPS is a bacterial NRPS. In some embodiments, the NRPS is a Streptomyces lavendulae NRPS (BpsA). In some embodiments, the NRPS comprises an amino acid sequence that is at least 70%, 80%, 90%, 95%, or 99% identical to the amino acid sequence of SEQ ID NO:1, wherein the NRPS comprises the enzymatic activity to convert glutamine to indigoidine.

The present invention provides for a method for a genetically modified yeast host cell producing indigoidine, comprising (a) providing a genetically modified yeast host cell of the present invention, (b) culturing or growing the host cell in a suitable culture or medium such that indigoidine is produced, and (c) optionally extracting or separating the indigoidine from the rest of the culture or medium, and/or host cell.

The present invention provides for a method for constructing a genetically modified yeast host cell of the present invention, comprising (a) introducing a nucleic acid encoding the NRPS operatively linked to a promoter capable of expressing the NRPS in the host cell into the host cell.

Indigoidine is a redox active blue pigment that has documented use as a dye. It can also serve as a respiration signal in cultivation optimizations and has the chemical structure of a molecule that can be used in development of biomaterials (e.g. polymers). The pigment may be used to report the redox and respiratory state of a large culture that may be critical for production performance. In some embodiments, the invention comprises the use of a heterologous codon-optimized version of an NRPS in an oleaginous yeast, the use of a wide set of renewable carbon sources (glucose, glycerol, hydrolysate) for the production of the pigment, and/or a very high titer production in a bioreactor scale (at least about 3-20 grams/liter).

### BRIEF DESCRIPTION OF THE DRAWINGS

The foregoing aspects and others will be readily appreciated by the skilled artisan from the following description of illustrative embodiments when read in conjunction with the accompanying drawings.

FIG. 1A. Production of bacterial indigoidine in engineered S. cerevisiae. S. cerevisiae exhibits two distinct metabolic states which are accompanied with distinct metabolic flux profiles. The width of the arrows represents metabolic flux. Blue arrows represent purely respiratory state, while red arrows represent fully fermentative state. GAP glyceraldehyde 3-phosphate, DHAP dihydroxyacetone phosphate, EtOH ethanol, α-KG α-ketoglutarate, Glu glutamate, Gln glutamine. Several known pathways for glutamine biosynthesis are shown. The depiction of metabolite intermediates and their cellular localization adapted from Frick et al. Ljungdahl and Daignan-Fornier, and Chen et al. [10, 48, 49].

FIG. 1B. Activation of the apo-form of the S. lavendulae NRPS, BpsA (blue pigment synthetase A) by the Bacillus subtilis 4′-phosphopantetheinyl transferase (PPTase; Sfp) via addition of a coenzyme A-derived moiety to the peptide carrier domain (PCP) into the active holo-form. The active holo-BpsA converts two 1-glutamines to one molecule of the blue pigment indigoidine by a catalytic process involving adenylation (a), oxidation (Ox) and thioesterase (TE) domains.

FIG. 1C. Positive S. cerevisiae transformants exhibit blue pigmentation occurring 3 days after visible colony formation on solid media containing glucose.

FIG. 1D. Brightfield microscopy of the pigmented colony shows heterogeneity in pigment production, ×63 zoom. The pigment shows punctate subcellular localization, scale bar=10 μm, increasing non-linear magnification of boxed areas is depicted by pull-outs.

FIG. 2A. Phenotype of BJ5465.sfp.bpsA grown on solid media containing glucose or glycerol. Colony color intensity of BJ5465.sfp.bpsA spotted on plates containing rich media and 2% glucose or the non-fermentable carbon source glycerol after 3 days and 7 days of growth. Colony color intensities are quantified using the Fiji image processing package distribution of ImageJ [45] and are normalized to highest detected colony intensity after brightness adjustment of the background. Error bars represent the standard deviation of 3 replicates. Representative colonies are shown in the panel below the graph.

FIG. 2B. Phenotype of BJ5465.sfp.bpsA grown on solid media containing glucose or glycerol. Bright field microscopy of cells grown on the non-fermentable carbon source glycerol after 3 days and 7 days, % blue represents the percentage of pigment producing cells of 500 cells counted for each condition, ×63 magnification, scale bar=10 μm.

FIG. 3A. Phenotype and Titer of BJ5465.sfp.bpsA grown in different carbon sources for 3 days. BJ5465.sfp.bpsA was grown in rich media containing glycerol ranging in concentrations from 1 to 5% as sole carbon source for 3 days. The carbon sources are utilized via different metabolic pathways in S. cerevisiae, namely respiratory for glycerol. Top: Quantification of indigoidine produced (blue bars) and remaining sugar in percentage (yellow bars) after 3 days of cultivation. Note difference in scale for indigoidine titer in glycerol compared to galactose and glucose. Middle: Quantification of ethanol (red bars), acetate (dark blue bars), and indigoidine (blue bars). Bottom: Representative photographs of respective liquid cultures after 3 days of cultivation. Error bars represent 95% CI (n=4).

FIG. 3B. Phenotype and Titer of BJ5465.sfp.bpsA grown in different carbon sources for 3 days. BJ5465.sfp.bpsA was grown in rich media containing galactose ranging in concentrations from 1 to 5% as sole carbon source for 3 days. The carbon sources are utilized via different metabolic pathways in S. cerevisiae, namely mixed respiro-fermentative for galactose. Top: Quantification of indigoidine produced (blue bars) and remaining sugar in percentage (yellow bars) after 3 days of cultivation. Note difference in scale for indigoidine titer in glycerol compared to galactose and glucose. Middle: Quantification of ethanol (red bars), acetate (dark blue bars), and indigoidine (blue bars). Bottom: Representative photographs of respective liquid cultures after 3 days of cultivation. Error bars represent 95% CI (n=4).

FIG. 3C. Phenotype and Titer of BJ5465.sfp.bpsA grown in different carbon sources for 3 days. BJ5465.sfp.bpsA was grown in rich media containing glucose ranging in concentrations from 1 to 5% as sole carbon source for 3 days. The carbon sources are utilized via different metabolic pathways in S. cerevisiae, namely fermentative for glucose. Top: Quantification of indigoidine produced (blue bars) and remaining sugar in percentage (yellow bars) after 3 days of cultivation. Note difference in scale for indigoidine titer in glycerol compared to galactose and glucose. Middle: Quantification of ethanol (red bars), acetate (dark blue bars), and indigoidine (blue bars). Bottom: Representative photographs of respective liquid cultures after 3 days of cultivation. Error bars represent 95% CI (n=4).

FIG. 4A. Cultivation profile of BJ5465.sfp.bpsA in different carbon sources. Concentrations of indigoidine (blue bars), consumed sugar (yellow line), dry cell weight (DCW, green line) and the by-products ethanol (red line) and acetate (dark blue line) are plotted against time for cells grown in glucose. Error bars represent 95% CI (n=4).

FIG. 4B. Cultivation profile of BJ5465.sfp.bpsA in different carbon sources. Concentrations of indigoidine (blue bars), consumed sugar (yellow line), dry cell weight (DCW, green line) and the by-products ethanol (red line) and acetate (dark blue line) are plotted against time for cells grown in glycerol. Error bars represent 95% CI (n=4).

FIG. 5A. Regulated environment in 2 L bioreactor enables control over metabolic state. Fed-batch fermentation of BJ5465.sfp.bpsA with excess glucose feed. Lines represent concentrations of total glucose fed and ethanol and acetate produced; bars represent indigoidine concentration. N=3 technical replicates for indigoidine extraction and DCW measurements.

FIG. 5B. Regulated environment in 2 L bioreactor enables control over metabolic state. Fed-batch fermentation of BJ5465.sfp.bpsA with signal-based pulse feeding strategy resulting in glucose starvation conditions. Lines represent concentrations of total glucose fed and ethanol and acetate produced; bars represent indigoidine concentration. N=3 technical replicates for indigoidine extraction and DCW measurements.

FIG. 6. R. toruloides was genetically engineered to produce the blue pigment indigoidine. Activation of the inactive apo-form of BpsA (blue pigment synthetase A) from S. lavendulae by Bacillus subtilis 4′-phosphopantetheinyl transferase Sfp (PPTase) via addition of a coenzyme A-derived moiety to the peptide carrier domain (PCP) into the active holo-form. The active holo-BpsA catalyzes a conversion of two L-glutamines to indigoidine involving adenylation (A), oxidation (Ox) and thioesterase (TE) domains.

FIG. 7A. Characterization of indigoidine production in R. toruloides (BlueBelle). Colonies of BlueBelle grown on agar plates exhibit blue pigmentation occurring with colony formation.

FIG. 7B. Characterization of indigoidine production in R. toruloides (BlueBelle). Cell pellet (P) and supernatant (S) show blue hue after separation of an indigoidine production culture by centrifugation.

FIG. 7C. Characterization of indigoidine production in R. toruloides (BlueBelle). Impact of filling volume on indigoidine production after 3 days of cultivation at 25° C. The colors used correspond to the respective cultivation temperature depicted in FIG. 7E. Cultivations in liquid culture were performed in synthetic defined media with a starting concentration of 100 g L⁻¹ glucose and 5 g L⁻¹ of ammonium sulfate.

FIG. 7D. Characterization of indigoidine production in R. toruloides (BlueBelle). Impact of cultivation temperature on growth, pH over the course of 5 days of cultivation. Error bars represent SD of 3 replicates. The colors used correspond to the respective cultivation temperature depicted in FIG. 7E. Cultivations in liquid culture were performed in synthetic defined media with a starting concentration of 100 g L⁻¹ glucose and 5 g L⁻¹ of ammonium sulfate.

FIG. 7E. Characterization of indigoidine production in R. toruloides (BlueBelle). Impact of cultivation temperature on indigoidine production after 3 days of cultivation using 5 mL filling volume. Error bars represent SD of 3 replicates. Cultivations in liquid culture were performed in synthetic defined media with a starting concentration of 100 g L⁻¹ glucose and 5 g L⁻¹ of ammonium sulfate.

FIG. 8. Colorimetric and chemical properties of indigoidine. Top The solution of indigoidine displays an increasingly intense hue of red when the pH decreases. Representative solutions are shown. Bottom Structural derivatives of indigoidine observed in this study shown in corresponding colors. Indigoidine can undergo hydrolysis to yield a red pigment, hydroxyidigoidine, which can react with NH₄OH to obtain indigoidine reversibly. Alternatively, hydroxyidigoidine can be deprotonated by NaOH and form a blue alkali metal adduct. In the presence of air, any form of indigoidine (i.e. indigoidine and hydroxyidigoidine) can be oxidized to form a ketone with a characteristic orange color.

FIG. 9. Impact of nitrogen source on the indigoidine production after 3 days of cultivation using 100 g L⁻¹ glucose. Nitrogen content was normalized to elemental nitrogen at a C/N ratio of 4. Error bars represent SD of 4 replicates.

FIG. 10. Impact of C/N ratio on the indigoidine production, microbial growth and culture pH after 3 days of cultivation using 100 g L⁻¹ glucose and varying amounts of urea as carbon and nitrogen source respectively. Differently colored circles in the table represent depictions of the culture hue. Error bars represent SD of 3 replicates.

FIG. 11A. Impact of carbon source on the indigoidine production. Metabolic pathways of R. toruloides to produce indigoidine from different carbon sources (yellow boxes), namely glucose, sucrose, glycerol and xylose. These pathways include glycolysis (grey), the pentose-phosphate-pathway (PPP, brown), the TCA-cycle (green) and central nitrogen metabolism (blue). Fatty acid and isoprenoid de novo synthesis pathways are shown in black.

FIG. 11B. Impact of carbon source on the indigoidine production. Concentrations of indigoidine (blue bars), consumed sugar (yellow line), OD800 (green line) and the culture pH (black rhombus) are plotted against time for cells grown in different carbon sources with an initial C/N ratio of 8 (starting sugar concentration was 100 g L⁻¹=10 g total in 100 mL), using urea as nitrogen source. Error bars represent SD of 3 replicates.

FIG. 12. Indigoidine production profile of BlueBelle using hydrolysate as carbon source. Concentrations of indigoidine (blue bars), consumed glucose (yellow line) and xylose (brown line), OD800 (green line) and the culture pH (black rhombus) are plotted against time for cells grown in hydrolysate obtained from different feedstocks (mixed feedstocks from Eucalyptus and switchgrass as well as solely from Eucalyptus) with an initial C/N ratio of 8, using urea as nitrogen source. The table shows glucose and xylose concentrations in media prepared with hydrolysates. Error bars represent SD of 3 replicates.

FIG. 13. Indigoidine production profile of BlueBelle in a separation-free 2 L bioreactor process. Concentrations of indigoidine (blue bars), consumed glucose (yellow line) and xylose (brown line) are plotted against time. Error bars represent 95% CI. Indigoidine extraction was performed in technical triplicates from the same sample.

FIG. 14. Indigoidine production profile of BlueBelle in a high-carbon fedbatch production process. Concentrations of indigoidine (blue bars), glucose consumed (yellow line) and OD800 (green line) are plotted against time. The arrow indicates the start of the adjusted feeding at increased rate on day 4. Error bars for indigoidine extraction represent SD from 3 technical triplicates.

### DETAILED DESCRIPTION OF THE INVENTION

Before the invention is described in detail, it is to be understood that, unless otherwise indicated, this invention is not limited to particular sequences, expression vectors, enzymes, host microorganisms, or processes, as such may vary. It is also to be understood that the terminology used herein is for purposes of describing particular embodiments only, and is not intended to be limiting.

In this specification and in the claims that follow, reference will be made to a number of terms that shall be defined to have the following meanings:

The terms “optional” or “optionally” as used herein mean that the subsequently described feature or structure may or may not be present, or that the subsequently described event or circumstance may or may not occur, and that the description includes instances where a particular feature or structure is present and instances where the feature or structure is absent, or instances where the event or circumstance occurs and instances where it does not.

As used in the specification and the appended claims, the singular forms “a,” “an,” and “the” include plural referents unless the context clearly dictates otherwise. Thus, for example, reference to an “expression vector” includes a single expression vector as well as a plurality of expression vectors, either the same (e.g., the same operon) or different; reference to “cell” includes a single cell as well as a plurality of cells; and the like.

In this specification and in the claims that follow, reference will be made to a number of terms that shall be defined to have the following meanings:

The terms “optional” or “optionally” as used herein mean that the subsequently described feature or structure may or may not be present, or that the subsequently described event or circumstance may or may not occur, and that the description includes instances where a particular feature or structure is present and instances where the feature or structure is absent, or instances where the event or circumstance occurs and instances where it does not.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

As used in the specification and the appended claims, the singular forms “a,” “an,” and “the” include plural referents unless the context clearly dictates otherwise. Thus, for example, reference to an “expression vector” includes a single expression vector as well as a plurality of expression vectors, either the same (e.g., the same operon) or different; reference to “cell” includes a single cell as well as a plurality of cells; and the like.

The term “about” refers to a value including 10% more than the stated value and 10% less than the stated value.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

The terms “host cell” is used herein to refer to a living biological cell that can be transformed via insertion of an expression vector.

The term “heterologous” as used herein refers to a material, or nucleotide or amino acid sequence, that is found in or is linked to another material, or nucleotide or amino acid sequence, wherein the materials, or nucleotide or amino acid sequences, are foreign to each other (i.e., not found or linked together in nature).

The terms “expression vector” or “vector” refer to a compound and/or composition that transduces, transforms, or infects a host cell, thereby causing the cell to express nucleic acids and/or proteins other than those native to the cell, or in a manner not native to the cell. An “expression vector” contains a sequence of nucleic acids (ordinarily RNA or DNA) to be expressed by the host cell. Optionally, the expression vector also comprises materials to aid in achieving entry of the nucleic acid into the host cell, such as a virus, liposome, protein coating, or the like. The expression vectors contemplated for use in the present invention include those into which a nucleic acid sequence can be inserted, along with any preferred or required operational elements. Further, the expression vector must be one that can be transferred into a host cell and replicated therein. Particular expression vectors are plasmids, particularly those with restriction sites that have been well documented and that contain the operational elements preferred or required for transcription of the nucleic acid sequence. Such plasmids, as well as other expression vectors, are well known to those of ordinary skill in the art.

The terms “polynucleotide” and “nucleic acid” are used interchangeably and refer to a single or double-stranded polymer of deoxyribonucleotide or ribonucleotide bases read from the 5′ to the 3′ end. A nucleic acid of the present invention will generally contain phosphodiester bonds, although in some cases, nucleic acid analogs may be used that may have alternate backbones, comprising, e.g., phosphoramidate, phosphorothioate, phosphorodithioate, or O-methylphophoroamidite linkages (see Eckstein, Oligonucleotides and Analogues: A Practical Approach, Oxford University Press); positive backbones; non-ionic backbones, and non-ribose backbones. Thus, nucleic acids or polynucleotides may also include modified nucleotides that permit correct read-through by a polymerase. “Polynucleotide sequence” or “nucleic acid sequence” includes both the sense and antisense strands of a nucleic acid as either individual single strands or in a duplex. As will be appreciated by those in the art, the depiction of a single strand also defines the sequence of the complementary strand; thus the sequences described herein also provide the complement of the sequence. Unless otherwise indicated, a particular nucleic acid sequence also implicitly encompasses variants thereof (e.g., degenerate codon substitutions) and complementary sequences, as well as the sequence explicitly indicated. The nucleic acid may be DNA, both genomic and cDNA, RNA or a hybrid, where the nucleic acid may contain combinations of deoxyribo- and ribo-nucleotides, and combinations of bases, including uracil, adenine, thymine, cytosine, guanine, inosine, xanthine hypoxanthine, isocytosine, isoguanine, etc.

The term “promoter,” as used herein, refers to a polynucleotide sequence capable of driving transcription of a DNA sequence in a cell. Thus, promoters used in the polynucleotide constructs of the invention include cis- and trans-acting transcriptional control elements and regulatory sequences that are involved in regulating or modulating the timing and/or rate of transcription of a gene. For example, a promoter can be a cis-acting transcriptional control element, including an enhancer, a promoter, a transcription terminator, an origin of replication, a chromosomal integration sequence, 5′ and 3′ untranslated regions, or an intronic sequence, which are involved in transcriptional regulation. These cis-acting sequences typically interact with proteins or other biomolecules to carry out (turn on/off, regulate, modulate, etc.) gene transcription. Promoters are located 5′ to the transcribed gene, and as used herein, include the sequence 5′ from the translation start codon (i.e., including the 5′ untranslated region of the mRNA, typically comprising 100-200 bp). Most often the core promoter sequences lie within 1-2 kb of the translation start site, more often within 1 kbp and often within 500 bp of the translation start site. By convention, the promoter sequence is usually provided as the sequence on the coding strand of the gene it controls. In the context of this application, a promoter is typically referred to by the name of the gene for which it naturally regulates expression. A promoter used in an expression construct of the invention is referred to by the name of the gene. Reference to a promoter by name includes a wildtype, native promoter as well as variants of the promoter that retain the ability to induce expression. Reference to a promoter by name is not restricted to a particular species, but also encompasses a promoter from a corresponding gene in other species.

A polynucleotide is “heterologous” to a host cell or a second polynucleotide sequence if it originates from a foreign species, or, if from the same species, is modified from its original form. For example, when a polynucleotide encoding a polypeptide sequence is said to be operably linked to a heterologous promoter, it means that the polynucleotide coding sequence encoding the polypeptide is derived from one species whereas the promoter sequence is derived from another, different species; or, if both are derived from the same species, the coding sequence is not naturally associated with the promoter (e.g., is a genetically engineered coding sequence, e.g., from a different gene in the same species, or an allele from a different ecotype or variety).

The term “operatively linked” refers to a functional relationship between two or more polynucleotide (e.g., DNA) segments. Typically, it refers to the functional relationship of a transcriptional regulatory sequence to a transcribed sequence. For example, a promoter or enhancer sequence is operably linked to a DNA or RNA sequence if it stimulates or modulates the transcription of the DNA or RNA sequence in an appropriate host cell or other expression system. Generally, promoter transcriptional regulatory sequences that are operably linked to a transcribed sequence are physically contiguous to the transcribed sequence, i.e., they are cis-acting. However, some transcriptional regulatory sequences, such as enhancers, need not be physically contiguous or located in close proximity to the coding sequences whose transcription they enhance.

The present invention provides for a genetically modified fungal host cell capable of producing indigoidine, wherein the host cell comprises a non-ribosomal peptide synthetase (NRPS) that converts glutamine to indigoidine.

In some embodiments, the fungal host cell is a yeast host cell. In some embodiments, the yeast host cell is a non-oleaginous yeast. In some embodiments, the yeast host cell is a non-oleaginous yeast. In some embodiments, the non-oleaginous yeast is a Saccharomyces species. In some embodiments, the Saccharomyces species is Saccharomyces cerevisiae. In some embodiments, the yeast host cell is an oleaginous yeast. In some embodiments, the oleaginous yeast is a Rhodosporidium species. In some embodiments, the Rhodosporidium species is Rhodosporidium toruloides.

In some embodiments, the NRPS is heterologous to the host cell. In some embodiments, the NRPS is a bacterial NRPS. In some embodiments, the NRPS is a Streptomyces lavendulae NRPS (BpsA). In some embodiments, the NRPS comprises an amino acid sequence that is at least 70%, 80%, 90%, 95%, or 99% identical to the amino acid sequence of SEQ ID NO:1, wherein the NRPS comprises the enzymatic activity to convert glutamine to indigoidine.

In some embodiments, the NRPS comprises a conserved domain, such as the amino acid sequence APRTETEKEI AEVWAKSLRR ESVSVQDDFF ESGGNSLIAV GLIRELNSRL GVSLPLQSVL ESPTVEKLSR RLEREV (SEQ ID NO:2), at the position corresponding to 937 to 1012 of SEQ ID NO:1.

In some embodiments, the NRPS comprises a coiled coil structure, such as the amino acid sequence SRRLEREV (SEQ ID NO:3), or SRRLEREVAQESSRLVRLHAE (SEQ ID NO:4), at the position corresponding to 1005 to 1012, or 1005 to 1025, of SEQ ID NO:1, respectively.

In some embodiments, the serine at position 972 is a modified serine, such as O-(pantetheine 4′-phosphoryl)serine.

The amino acid sequence of Streptomyces lavendulae BpsA is as follows:

```
(SEQ ID NO: 1)
                10 20 30 40
MTLQETSVLE PTLRGTTTLP DLLAKRVAEH PEATAVAYRD
                50 60 70 80
EKLTYRELAS RSSALAEYLR HLGVSTDDCV GLFVEPSIDL
                90 100 110 120
MVGAWGILSA GAAYLPLSPE YPEDRLRYMI ENSQAKIILA
              130 140 150 160
QQRLVTRLRE LAPQDVRVVT LRESEAFVLP EGQVAPAIEG
              170 180 190 200
ARPDSLAYVI YTSGSTGKPK GVMIEHHSIV SQLGWLRETY
              210 270 230 240
GIDRSKTILQ KTPMSFDAAQ WEILSPANGA TVVMGAPGVY
              250 260 270 280
ADPEGLIETI VKYGVTTLQC VPTLLQGLLD TEKFPECTSL
              290 300 310 320
QQIFSGGEAL SRLLAIQTTQ EMPGRALINV YGPTECTINS
              330 340 350 360
SSYAVDPAEL GEAPQSISIG APVADTEYHI LGKEDLKPVG
              370 380 390 400
VGEIGELYIG GGQLARGYLH RPDLTAERFL EIEVTEGAGP
              410 420 430 440
VRLYKTGDLG QWNPDGTVQF AGRADNQVKL RGYRVELDEI
              450 460 470 480
SLAIENHDWV RNAAVIVKND GRTGFQNLIA CVELSEKEAA
              490 500 510 520
LMDQGNHGSH HASKKSKLQV KAQLSNPGLR DDADLAARVA
              530 540 550 560
YDLPGAEPTP EQRSRVFARK TYRFYEGGAV TEADLLALLG
              570 580 590 600
GQVPAAYSRK AADLAPAELG QTLRWFGQYL SEERLLPKYG
              610 620 630 640
YASPGALYAT QLYFELEGVG GLQPGYYYYQ PQRHQLVLIS
              650 660 670 680
EKAATGRPTA HIHFIGKRGG IEPVYKNNIQ EVLEIETGHI
              690 700 710 720
VGLFEQVLPA YGLDIRDLAY EPAVRDLLDV PEEDFYLGTF
              730 740 750 760
ELVPHTGRRE DRAEVYVQTH GSKVANLPEG QYRYADGTLT
              770 780 790 800
RFSDDIVLKK QVIAINQSVY QAASFGISVI SRAPEEWMHY
              810 820 830 840
VTLGKKLQHL MMNGLGLGFM SSGYSSKTGN PLPASRRIDS
              850 860 870 880
VLQANGVESG PSYFFVGGRV SDEQLGHEGM REDSVHMRGP
              890 900 910 920
AELIRDDLVS FLPDYMIPNR VVVFERLPLS ANGKIDAKAL
              930 940 950 960
AASDQVNAEL VERPFVAPRT ETEKEIAEVW AKSLRRESVS
              970 980 990 1000
VQDDFFESGG NSLIAVGLIR ELNSRLGVSL PLQSVLESPT
            1010 1020 1030 1040
VEKLSRRLER EVAQESSRLV RLHAETGKDR PVLCWPGLGG
            1050 1060 1070 1080
YPMNLRTLAG EIGLGRSFYG IQAHGINEGE APYATITEMA
            1090 1100 1110 1120
KADIEAIKEL QPKGPYTLWG YSFGARVAFE TAYQLEQAGE
            1130 1140 1150 1160
KVDNLFLIAP GSPTVRAENG KVYGREASFA NRAYTTTLFS
            1170 1180 1190 1200
VFTGTISGPD LEKCLESATD EESFAGFISE LKGIDVDLAK
            1210 1220 1230 1240
RIISVVGQTY EFEYSFRELA ERTLAAPVTI FKARGDDYSF
            1250 1260 1270 1280
IENSNGYSAE PPTVIDLDAD HYSLLRTPDI GELVKHIRYL
LGE
```

In some embodiments, the host cell comprises a nucleic acid encoding the NRPS operatively linked to a promoter capable of expressing the NRPS in the host cell. In some embodiments, the encoding of the NRPS to the nucleic acid is codon optimized to the yeast host cell. In some embodiments, the nucleic acid is vector or replicon that can stably reside in the host cell. In some embodiments, the nucleic acid is stably integrated into one or more chromosomes of the host cell.

The present invention provides for a method for a genetically modified yeast host cell producing indigoidine, comprising (a) providing a genetically modified yeast host cell of the present invention, (b) culturing or growing the host cell in a suitable culture or medium such that indigoidine is produced, and (c) optionally extracting or separating the indigoidine from the rest of the culture or medium, and/or host cell.

In some embodiments, the providing step (a) comprises introducing a nucleic acid encoding the NRPS operatively linked to a promoter capable of expressing the NRPS in the host cell into the host cell.

In some embodiments, the culturing or growing step (b) comprises the host cell growing by respiratory cell growth. In some embodiments, the culturing or growing step (b) takes place in a batch process or a fed-batch process, such as a high-gravity fed-batch process. In some embodiments, the culture or medium comprises hydrolysates derived or obtained from a biomass, such as a lignocellulosic biomass. In some embodiments, the culture or medium comprises one or more carbon sources, such as a sugar, such as glucose or galactose, or glycerol, or a mixture thereof. In some embodiments, the carbon source is fermentable. In some embodiments, the carbon source is non-fermentable.

In some embodiments, the culture or medium comprises urea as a nitrogen course. In some embodiments, the culture or medium comprises urea for at least about 20%, 30%, 40%, 50%, 60%, 70%, 80%, or 90% of the nitrogen source for the host cell.

In some embodiments, the culturing or growing step (b) is capable of producing up to about 50, 100, 110, 120, 130, 140, 150, 160, 170, 180, 190, 200, or 210 mg of indigoidine per liter of culture or medium, or a value within a range of any preceding two values. In some embodiments, the host cell is a Saccharomyces species, such as Saccharomyces cerevisiae.

In some embodiments, the culturing or growing step (b) is capable of producing up to about 0.1, 0.2, 0.3, 0.4, 0.5, 0.6, 0.7, 0.8, 0.9, 1.0, 1.1, 1.2, 1.3, 1.4, 1.5, 2, 3, 4, 5, 6, 7, 8, 9, 10, 11, 12, 13, 14, 15, 16, 17, 18, or 19 g of indigoidine per liter of culture or medium, or a value within a range of any preceding two values. In some embodiments, the host cell is a Rhodosporidium species, such as Rhodosporidium toruloides.

The present invention provides for a method for constructing a genetically modified yeast host cell of the present invention, comprising (a) introducing a nucleic acid encoding the NRPS operatively linked to a promoter capable of expressing the NRPS in the host cell into the host cell.

In some embodiments, this invention comprises of an engineered Rhodosporidium toruloides strain containing a heterologously expressed, yeast codon-optimized, non-ribosomal peptide synthetase (NRPS) gene bpsA from Streptomyces lavendulae, leading to the production of high levels the blue pigment indigoidine. Rhodosporidium toruloides is a valuable new microbial platform that can convert a diverse set of renewable carbon sources to final products. While the pathway for indigoidine production is known and has also been heterologously expressed in model hosts such as E. coli, this is the first use of a yeast codon optimized version of the enzyme in an engineered fungal host that can utilize a large number of renewable intermediates. Indigoidine has many commercial uses, including as a dye. The present invention can produce a very high titer for the dye: such as 3-20 grams/liter from both fermentable (glucose) and non-fermentable (glycerol) carbon sources, and 0.15-0.35 grams/liter from two different types of saccharification broths. The present invention also provides for an engineered Rhodosporidium toruloides strain containing a heterologous expressed, yeast codon-optimized, non-ribosomal peptide synthetase (NRPS) gene bpsA, leading to the production of the blue dye indigoidine at least about 3-20 grams/liter from both fermentable (glucose) and non-fermentable (glycerol) carbon sources, and at least about 0.15-0.35 grams/liter from two different types of saccharification broths.

One can modify the expression of a gene encoding any of the enzymes taught herein by a variety of methods in accordance with the methods of the invention. Those skilled in the art would recognize that increasing gene copy number, ribosome binding site strength, promoter strength, and various transcriptional regulators can be employed to alter an enzyme expression level.

It is to be understood that, while the invention has been described in conjunction with the preferred specific embodiments thereof, the foregoing description is intended to illustrate and not limit the scope of the invention. Other aspects, advantages, and modifications within the scope of the invention will be apparent to those skilled in the art to which the invention pertains.

All patents, patent applications, and publications mentioned herein are hereby incorporated by reference in their entireties.

The invention having been described, the following examples are offered to illustrate the subject invention by way of illustration, not by way of limitation.

### Example 1

### Production Efficiency of the Bacterial Non-Ribosomal Peptide Indigoidine Relies on the Respiratory Metabolic State in S. cerevisiae

Background: Beyond pathway engineering, the metabolic state of the production host is critical in maintaining the efficiency of cellular production. The biotechnologically important yeast Saccharomyces cerevisiae adjusts its energy metabolism based on the availability of oxygen and carbon sources. This transition between respiratory and non-respiratory metabolic state is accompanied by substantial modifications of central carbon metabolism, which impact the efficiency of metabolic pathways and the corresponding final product titers. Non-ribosomal peptide synthetases (NRPS) are an important class of biocatalysts that provide access to a wide array of secondary metabolites. Indigoidine, a blue pigment, is a representative NRP that is valuable by itself as a renewably produced pigment.

Results: Saccharomyces cerevisiae was engineered to express a bacterial NRPS that converts glutamine to indigoidine. We characterize carbon source use and production dynamics, and demonstrate that indigoidine is solely produced during respiratory cell growth. Production of indigoidine is abolished during non-respiratory growth even under aerobic conditions. By promoting respiratory conditions via controlled feeding, we scaled the production to a 2 L bioreactor scale, reaching a maximum titer of 209.9 mg/L.

Conclusions: This study represents the first use of the Streptomyces lavendulae NRPS (BpsA) in a fungal host and its scale-up. The final product indigoidine is linked to the activity of the TCA cycle and serves as a reporter for the respiratory state of S. cerevisiae. Our approach can be broadly applied to investigate diversion of flux from central carbon metabolism for NRPS and other heterologous pathway engineering, or to follow a population switch between respiratory and non-respiratory modes.

### Background

Microbial metabolic pathway discovery and engineering efforts have led to an increasing number of biotechnological processes in diverse sectors of our economy, ranging from energy to health and medicine, as well as food and agriculture. Industrial-scale microbial production environments are profoundly different from the cultivation environments commonly used at lab scale. Thus, beyond pathway engineering, understanding microbial physiology in these different environments is essential to translate proof-of-concept bioprocesses from shake flasks to industrially-relevant bioreactor setups [1, 2]. During large-scale biotechnological production processes, insufficient mixing commonly leads to micro-environmental inhomogeneities with severe concentration gradients of important cultivation characteristics, particularly dissolved oxygen and carbon sources [3]. The benefit of using facultative anaerobic microbes in industrial processes arises from their ability to switch between fermentative and respiratory metabolism to produce ATP depending on the availability of oxygen without loss of viability. However, fluctuations in dissolved oxygen and carbon sources are recognized to trigger metabolic and transcriptional responses, with unfavorable effects on productivity [2, 3, 4].

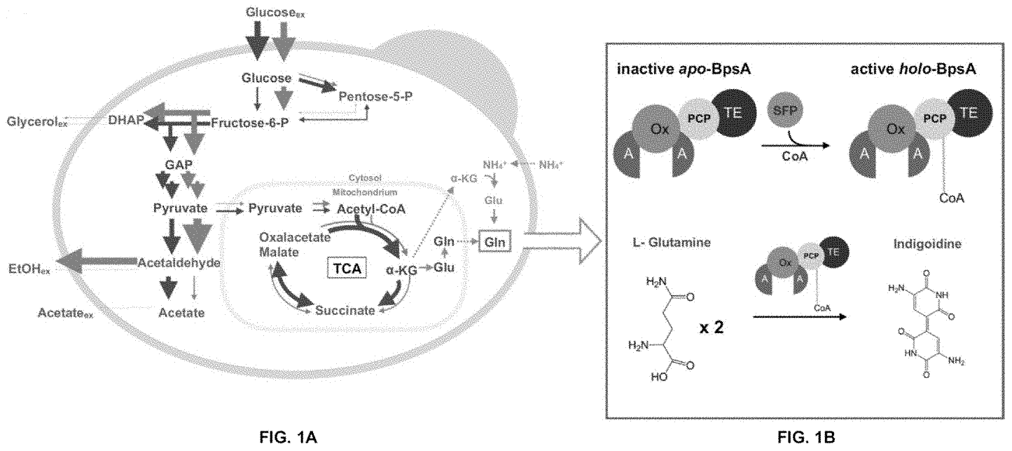

Saccharomyces cerevisiae is not only used extensively for proof-of-concept pathway studies but also as a host for many applied industrial processes [5, 6]. In contrast to many other fungal or bacterial hosts, S. cerevisiae adjusts its energy metabolism based on the nature of available carbon sources via carbon catabolite repression [7]. Even under aerobic conditions, S. cerevisiae predominantly metabolizes glucose by fermentation leading to the production of ethanol, glycerol and carbon dioxide (FIG. 1A, red arrows) [8, 9]. Upon glucose depletion, the non-fermentable products of fermentation ethanol and glycerol can serve as carbon sources, requiring a shift to respiratory mode. The metabolic shift from fermentative to respiratory growth is accompanied by changes of carbon flux and gene expression throughout the whole central metabolism [10, 11]. Under purely fermentative conditions, a redirection of metabolic flux from the tricarboxylic acid (TCA) cycle towards fermentative pathways results in a low activity of the TCA cycle. When switching from fermentative to respiratory conditions, the flux to the TCA cycle increases significantly to enable respiration (FIG. 1A, blue arrows) [10, 12, 13]. Thus, activity of the TCA cycle presents an appropriate proxy to distinguish metabolic states in S. cerevisiae [14]. While the effect of the metabolic state on native pathways and products has been investigated [15, 16, 17, 18], its effect on engineered pathways and biosynthetic products remains understudied.

In this study, we engineered S. cerevisiae for the production of indigoidine, a non-ribosomal peptide synthetase (NRPS) derived compound, formed by condensation of two 1-glutamine residues. Specifically, we use the bacterial Blue pigment synthetase (BpsA) from Streptomyces lavendulae [19], that has not been expressed in a fungal host before. Non-ribosomal peptides present a diverse class of secondary metabolites with various important biological activities. Indigoidine itself, provides a renewably produced pigment for the dye industry that has reinvigorated its search for environmentally friendly processes [20]. Indigoidine is an ideal final heterologous product to examine the importance of respiratory and non-respiratory environments as its precursor pool is linked to the TCA cycle. We examine the effect of the metabolic state on this heterologous product derived from the TCA cycle, a pathway highly responsive to metabolic shifts. Using colorimetric production assays and metabolomics, we demonstrated that the production of the indigoidine is connected to the metabolic state of the cell and can be maintained with high fidelity if S. cerevisiae is kept in respiratory mode. Further, we use this knowledge to maintain high levels of indigoidine production when transitioning between cultivation format and scales.

### Results and Discussion

Establishing Indigoidine Production in Saccharomyces cerevisiae

In S. lavendulae, the native pathway to convert 1-glutamine into the blue pigment indigoidine consists of the NRPS BpsA and a 4′-phosphopantetheinyl transferase (PPTase), needed to activate the apo-NRPS into its holo-form via the addition of a coenzyme A-derived phosphopantetheine moiety (FIG. 1B) [19, 21]. To establish the indigoidine pathway in S. cerevisiae, we genomically integrated the Bacillus subtilis PPtase sfp, previously shown to successfully activate apo-BpsA [22] and the 3.8 kbp NRPS gene bpsA into S. cerevisiae BJ5465, a protease deficient strain reported to functionally express Sfp [23].

Blue pigment production was successfully observed in the resulting strain 3 days after visible colony formation (FIG. 1C). The pigmentation appeared first in the central colony region and was limited to subpopulations on the colony surface and extended outwards of the colony over the course of 10 days. This observation indicates that the localization of a given cell within a colony has an effect on the production. This effect could originate from enhanced oxygen availability at the surface of the central colony region as compared to the outer limits or lower layers of a colony [24], as oxygenation is a necessary step in the formation of the pigment [25].

To determine the localization of the pigment within the cell, we performed brightfield microscopy of the transformants. As expected from the phenotype of the colony, the population shows heterogeneity regarding pigment production (FIG. 1D). In cells that produce the blue pigment, it accumulates in foci and forms aggregates.

### Carbon Source Determines Efficiency of Indigoidine Production

While glucose is its preferred carbon source, S. cerevisiae can utilize other sugars such as sucrose, galactose and a variety of non-fermentable substrates including glycerol by adjusting its energy metabolism from fermentation to respiration. The flux through the TCA cycle is significantly increased during respiration compared to that during fermentation (FIG. 1A) [10]. The TCA cycle intermediate alpha-ketoglutarate serves as an indirect precursor pool for indigoidine formation via the amino acids glutamate and glutamine.

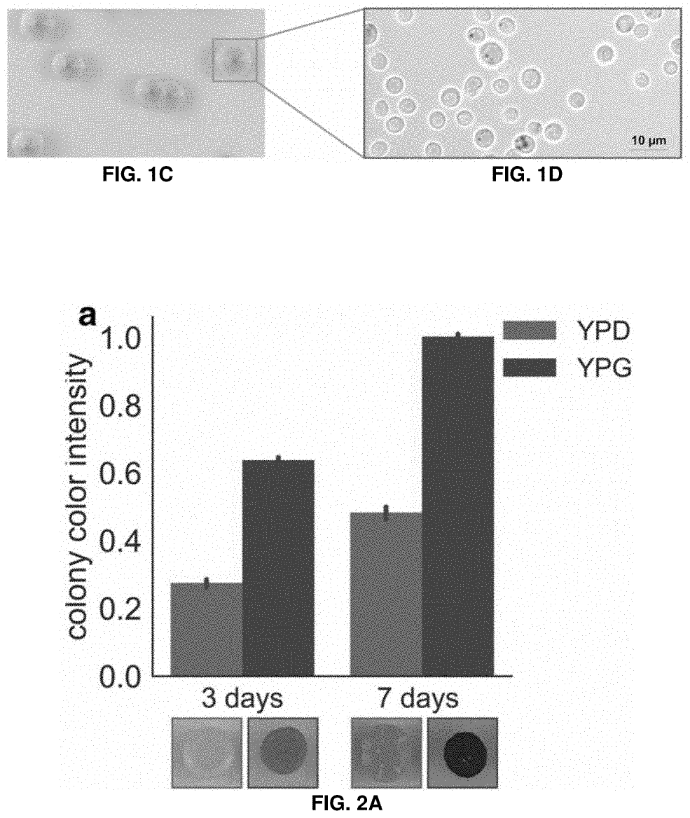

Thus, we hypothesized that efficient formation of indigoidine as a product of the TCA cycle takes place predominantly during the respiratory metabolic state and not during fermentative growth. To test this hypothesis, BJ5465.sfp.bpsA was grown on solid rich media containing either 2% glucose or 2% glycerol as sole carbon sources and pigment formation was monitored. When grown on medium containing glycerol, visible blue pigmentation coincided with visible colony formation after 3 days of incubation at 37° C. and increased in intensity to reach maximum pigmentation after additional 4 days (FIG. 2A). Using glucose as a carbon source caused a delay in visible pigmentation but increased growth rate of the colonies as compared to glycerol. Because glycerol is a non-fermentable carbon source, cells are required to shift into respiratory metabolic state, which leads to a decrease in growth rate but an increased flux through the TCA cycle. Furthermore, blue pigment production was absent in spontaneous petite mutants grown on medium containing 2% glucose, indicating the requirement of functional mitochondria for indigoidine formation. Petite mutants form small colonies on fermentable carbon sources and are unable to grow on non-fermentable carbon sources due to absent or dysfunctional mitochondria and thus TCA cycle deficiency [26]. Therefore, these observations are consistent with our hypothesis that efficient production of indigoidine occurs during respiratory growth.

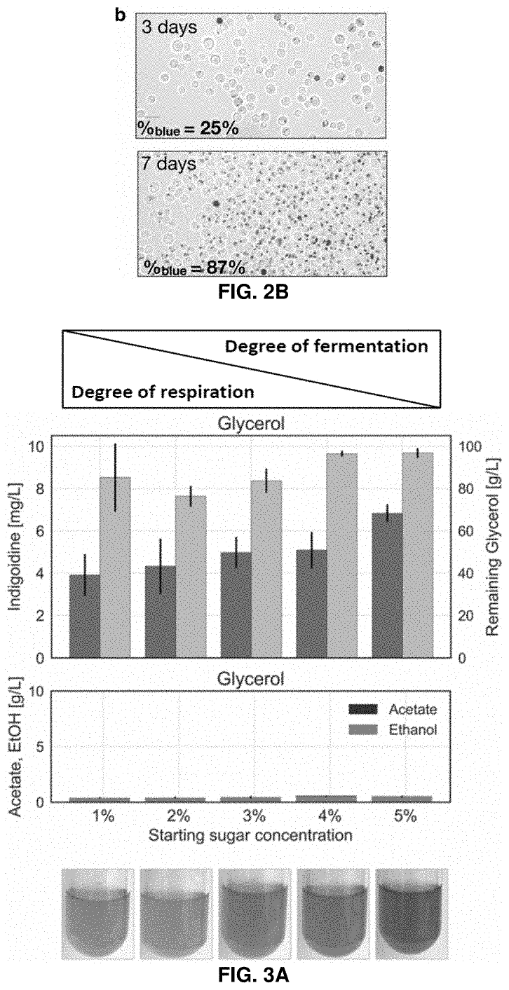

For sucrose and glucose, pigment production was observed at high titers for low starting sugar concentrations of 1% and 2% but was absent at 4% and 5% initial sugar concentrations, while the by-products ethanol and acetate were detected in increasing amounts with increasing starting sugar concentrations (FIG. 3C).

We hypothesized that the lack of pigment production at higher starting concentrations of these fermentable carbon sources after 3 days could be caused by remaining sugar. Unconsumed sugar present in the medium at sufficient concentrations could cause the cells to remain in the fermentative state, inhibiting flux through the TCA cycle and thus preventing indigoidine production. To test this hypothesis, we performed sugar and by-product quantification using HPLC. This analysis revealed that 99% of the sugar is consumed independent of starting glucose concentrations (FIG. 3C, middle), rendering excess sugar as cause for the absence of pigment production unlikely. This conclusion is supported by the observation that pigment production remained absent in cultures of 4% or 5% glucose or sucrose concentration even after additional 48 h of cultivation. The absence of pigment production in cultures with high initial sugar concentrations could originate from nitrogen limitations of these cultures at later stages of their growth. This conclusion is in agreement with results from observations made by Brown and Johnson [27] when analyzing the effect of sugar concentrations on cell yield and metabolites of S. cerevisiae cultures.

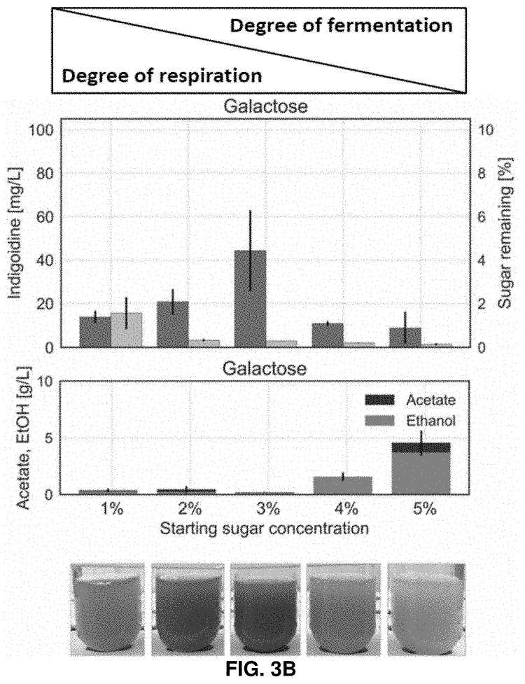

To gain a detailed understanding of the production profile, we captured the dynamics of metabolite abundances (carbon source, ethanol, acetic acid) and quantified pigment production over the course of 4 days. As expected, the carbon consumption profile of BJ5465.sfp.bpsA grown in medium containing glucose resembles a typical profile for aerobic diauxic growth by S. cerevisiae [29]. In the first 24 h of cultivation, glucose was fully consumed by fermentative metabolism resulting in the production of 6.96∓0.85 g/L ethanol, 0.07∓0.01 g/L acetate and biomass accumulation of 5.13∓0.78 g/L (FIG. 4A). In a subsequent respiratory metabolic growth phase, the non-fermentable carbon source ethanol was consumed leading to slower biomass formation. The shift from glucose consumption to ethanol consumption, marks the onset of indigoidine production after 24 h. These results indicate that indigoidine production coincided with the shift from fermentative to respiratory metabolism for cells grown on glucose containing medium. Thus, we expected that growth on a non-fermentable carbon source would eliminate the delay in indigoidine production caused by an initial fermentative growth phase. Indeed, growth on glycerol resulted in prompt production of blue pigment (FIG. 4B), even though glycerol was consumed at very slow rates throughout the experiment. Ethanol and acetate were produced at negligible amounts during the entire growth phase as expected for respiratory growth. In contrast to growth on glucose, indigoidine production profile during growth on glycerol correlated with the biomass profile.

### Altering Metabolic States Through Controlled Carbon Availability in Bioreactors can Enhance Indigoidine Production

Advanced process control available in bioreactors can be used to influence microbial growth and product generation through controlled culture environments. Our previous experiments were performed in tubes and shake flasks in batch-mode, where no additional substrate was added after the start of the cultivations. In these batch fermentations, substrate depletion affected the metabolic state of the cultures. To maintain a specific metabolic state over an extended period of time, fed-batch cultivations with two different substrate feeding strategies, i.e. carbon depletion and carbon excess, were performed in 2 L bioreactors.

Carbon depletion conditions were implemented using a dissolved oxygen (DO) signal-based pulse feeding strategy. The metabolic activity of cells stalls upon depletion of total carbon in the culture. First, glucose is fully consumed, followed by the consumption of other sources of carbon such as fermentative by-products ethanol and acetate. The stall in metabolic activity leads to a reduction in oxygen demand, resulting in a sudden increase (“spike”) of dissolved oxygen levels in the culture. A “pulsed” feed of glucose was triggered upon carbon depletion events detected by a DO spike. Excess availability of carbon was achieved through semi-continuous feeding of glucose with fixed delivery of 4 g/L/h.

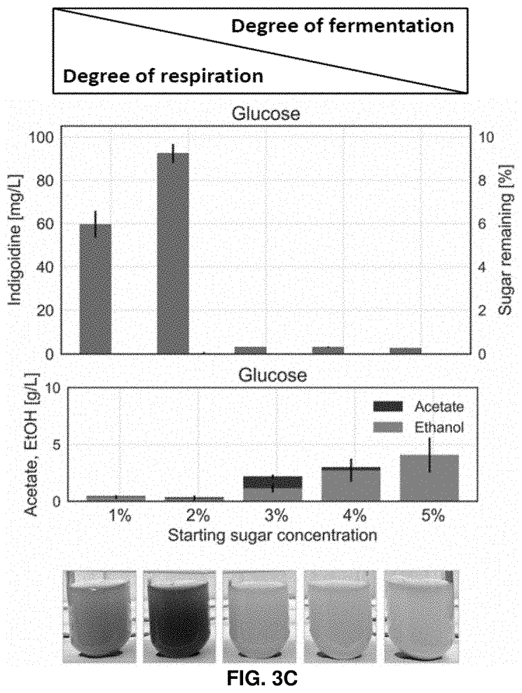

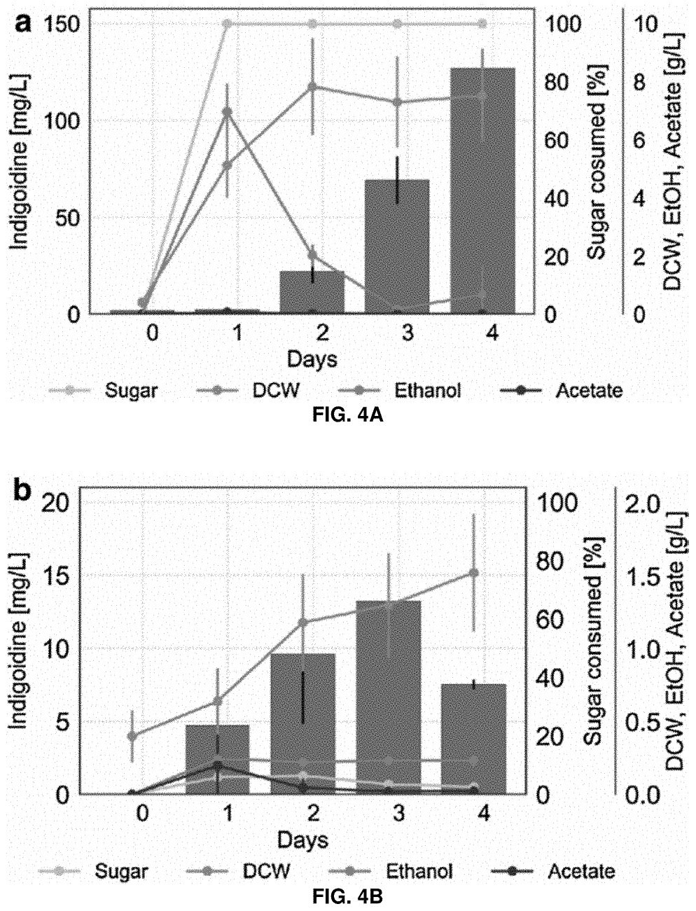

We hypothesized that excess carbon conditions would promote fermentative metabolism while carbon depletion would enable respiratory metabolism. Indeed, excess conditions resulted in accumulation of the by-products ethanol and acetate reaching final concentrations of 55.3 g/L and 3.1 g/L, respectively (FIG. 5A). No significant production of the pigment was observed. These observations agree with our hypothesis that fermentative metabolic state and thus the inactivity of the TCA cycle impedes efficient pigment formation.

In contrast, depletion conditions resulted in a high production titer of indigoidine, reaching 209.9 mg/L at the end of the fermentation while accumulating only negligible amounts of ethanol and acetate throughout (FIG. 5B). Interestingly, excess availability of glucose did not have a significant effect on biomass formation as determined by dry cell weight. These results indicate that the growth parameters selected for glucose starvation conditions imposed a predominantly respiratory metabolic state leading to the activation of the TCA cycle and production of indigoidine.

### Conclusion

Our findings demonstrate that the metabolic state of the cell is critical for the efficiency of a biosynthetic pathway. We showed that production of the NRPS catalyzed blue pigment indigoidine, a product of the TCA cycle, is linked to the respiratory metabolic state in S. cerevisiae. Important cultivation parameters, known to affect the metabolic state of S. cerevisiae, shape the indigoidine production profile regarding timing and titer. In the case of non-fermentable carbon sources that are consumed via respiratory metabolism, pigment production occurs at the same time as biomass formation (e.g. DCW). In contrast, growth on glucose results in a delay of indigoidine production until after the glucose is consumed.

Our results are consistent with¹³C metabolic flux studies that report redirection of flux towards the TCA cycle during respiratory metabolism compared to fermentation [10, 12]. While it is known that several native pathways undergo a redirection of flux accompanying the shift from fermentative to respiratory metabolism in S. cerevisiae [11, 18, 30], these aspects are rarely considered during initial strain engineering or demonstration of production. However, as shown in this study, changes in the metabolic flux profile and precursor pools do have major implications for the productivity of the host cell. In this context, the TCA cycle is specifically important as it is not only the main pathway for generation of reducing equivalents, but also produces important intermediates and precursors for biosynthetic products such as amino acids from the aspartate and alpha-Ketoglutarate families [31, 32] and short-chain dicarboxylic acids such as succinate [33, 34]. In addition to the TCA cycle, metabolic flux of other pathways, commonly employed in metabolic engineering, like the pentose phosphate pathway and the glyoxylate cycle have also been shown to be affected by metabolic redirections [10, 12].

The dependence of metabolic state and production efficiency of biosynthetic pathways becomes increasingly important when transitioning into industrial scale production or fed-batch mode. In these conditions, insufficient mixing commonly leads to heterogeneity in substrate and oxygen distribution E. Indeed, Fu et al. [35] reported a prominent difference in glucose catabolism in S. cerevisiae in response to transitioning from laboratory (10 L) to industrial scale (10,000 L), and detected a loss of TCA cycle intermediates through secretion relating to mitochondrial dysfunction at industrial as compared to laboratory scale. Our findings underscore the need for identification of production strains that maintain robust performance in the presence of large concentration gradients over the course of a production process with in a bioreactor. In addition to constructing strains with the desired biosynthetic pathway, several strain attributes and host selection criteria need to be considered a priori, for a given final product, to effectively develop engineered microbes well-suited for large-scale aerobic cultivation [30]. Given the growing potential of metabolic engineering tools available, a solution to this problem can be seen in “rewiring” the central carbon metabolism to increase the energy efficiency of the production pathway of a given production strain and thereby reducing the oxygen demand, for example to increase the efficiency of acetyl CoA-based isoprenoid production in S. cerevisiae [37].

To our knowledge, this is the first report of a high-titer production of the non-ribosomal peptide indigoidine in a fungal host, achieving 209.9 mg/L indigoidine at a 2 L bioreactor scale. We demonstrated that indigoidine formation is linked the respiratory metabolic state in S. cerevisiae and maintenance of the required metabolic state was critical for enhancing its production levels at higher scales [38, 39]. Our study illustrates that a better understanding of the metabolic states involved in heterologous production in the respective production environment is imperative for a reliable outcome in strain performance and has to be taken into consideration during strain engineering. In addition to contributing to understanding the importance of the metabolic state of the production host for optimal performance in bioprocesses, our system may be used as a control for metabolic state during strain and process development.

### Materials and Methods

### Strain Construction

All S. cerevisiae strains used in this study are derived from the protease deficient strain BJ5465: MATa ura3-52 trp1 leu2-Δ1 his3-δ200pep4::HIS3 prb1-δ1.6R can1 GAL (ATCC). All strains and strain information have been deposited in the public instance of the JBEI Registry [40] (website for: public-registry.jbei.org/folders/386) and are physically available from the authors upon request.

To create the strain BJ5465.sfp.bpsA, sfp was integrated into the yeast chromosomal 6-sequences [41]. The bpsA gene was codon-optimized for expression in S. cerevisiae (Genscript, Piscataway N.J.) and genomically integrated into locus ARS1014a under control of the TDH3 promoter and ADH1 terminator using a previously reported, cloning free Cas9 toolkit [42]. Transformations were performed using the conventional lithium acetate method [43] using 200 ng pCut_1014a and 500 ng of linear Donor DNA with 500 bp homology to the integration locus ARS1014a.

E. coli strain Bap1 [44] was transformed with a E5C plasmid encoding bpsA codon-optimized for expression S. cerevisiae and used as a host to establish indigoidine production and prepare a standard curve for quantification of pigment production.

### Media and Cultivation Conditions

Overnight cultures of S. cerevisiae were grown in 5 mL standard rich Glucose medium (YPD, 1% (w/v) Bacto yeast extract, 2% (w/v) Bacto peptone, 2% (w/v) Dextrose) at 30° C., shaking at 200 rpm. Production cultures were inoculated to an OD₆₀₀ of 0.05 in rich medium [YP, 1% (w/v) Bacto yeast extract, 2% (w/v) Bacto peptone and 2% (w/v) Sugar], unless stated otherwise and grown at 30° C. at 200 rpm. All productions were carried out in quadruplets.

### Imaging and Color Intensity Quantification

Pictures of plates and culture tubes were taken with a 12-megapixel camera. The means of color intensity of three colonies was quantified using the Fiji image processing package distribution of ImageJ [45]. For this analysis, the colorization of the plates was adjusted to match, based on background color. The analysis was carried out for three different colonies from three technical replicates each.

For brightfield microscopy, 1 μL of cells from liquid culture or an equivalent of 1 μL from colonies grown on agar plates were imaged for blue pigment production studies using a Leica-DM4000B microscope equipped with a Hamamatsu Digital Camera C4742-95 and a Micropublisher 5.0 RTV Camera with a 63× or 100× objective and processed using Leica software (Leica Application Suite X, LAS X). To determine the ratio of pigment producing cells of cells in a population, 500 cells each were counted and categorized from microscopy pictures.

### Indigoidine Extraction

Purification of indigoidine was performed using a modified protocol from Yu et al. [46]. Briefly, 1 mL of culture was centrifuged at 21,000×g for 3 min and the supernatant removed. To lyse the cells and extract indigoidine, 100 μL of acid washed beads (625 nm) and 1 mL DMSO+2% Tween® 20 were added to the cell pellet and vortexed twice for 1 min using Mini-Beadbeater-96 (Biospec, Bartlesville Okla.) at 3600 rpm. After centrifugation at 21,000×g for 3 min, the indigoidine concentration was determined by measuring the OD₆₁₂ of the supernatant using a BioTek Synergy 4 plate reader (Biotek, Winooski Vt.), preheated to 25° C. and applying a standard curve.

### Preparation of Indigoidine Standard Curve

The E. coli strain Bap1 E5C.bpsA was grown overnight at 37° C. in 5 mL LB medium (Beckton Dickinson, NJ, USA; Cat. No. 244610) supplemented with 25 μg/mL Chloramphenicol and back diluted to OD₆₀₀ of 0.1 in 10 mL LB Chloramphenicol the next morning. The strain was cultivated at 37° C. shaking at 200 rpm to reach OD600 of 0.4, induced with 1 mM isopropyl-1-thio-β-d-galactopyranoside (IPTG, Sigma-Aldrich, St. Louis Mo.) and further cultivated at 30° C., 200 rpm for 24 h before harvesting the cell pellet by centrifugation 10,000×g for 5 min. To lyse the cells and extract indigoidine, 100 μL of acid washed beads (625 nm) and 1 mL DMSO+2% Tween® 20 were added to the cell pellet and vortexed twice for 1 min using Mini-Beadbeater-96 (Biospec, Bartlesville Okla.) at 3600 rpm. The mixture was centrifuged, and the supernatant was dried in vacuo. To obtain pure indigoidine, the resulting pellet was washed twice each with 1 mL water, 1 mL EtOAc, 1 mL MeOH and 1 mL Hexane and dried again in vacuo. Afterwards, 0.64 mg of dried indigoidine was dissolved in 1 mL DMSO. This solution was further serially diluted into six different concentrations (0.01, 0.02, 0.04, 0.08, 0.16, 0.32 mg/mL) and measured for OD₆₁₂ values using a BioTek Synergy 4 plate reader, preheated to 25° C. The standard curve was established by the linear relationship between the absorbance and concentration according to Kuhn et al. [47].

### Quantification of Sugar, Ethanol and Acetate

Sugar and by-product concentrations were quantified on a 1200 series HPLC (Agilent Technologies) equipped with an Aminex H column (Bio-Rad, Hercules Calif.). Samples were filtered through 0.45 μm filters (VWR) to remove cells, and 5 μL of each sample was injected onto the column, preheated to 50° C. The column was eluted with 4 mM H₂SO₄ at a flow rate of 600 μL/min for 25 min. Sugars and metabolites were monitored by a refractive index detector, and concentrations were calculated by peak area comparison to known standards.

### Fed-Batch Experiments at 2 L Bioreactor Scale

Fed-batch experiments were performed using 2 L Sartorius BIOSTAT B® fermentation system (Sartorius AG., Goettingen, Germany), each agitated with two Rushton impellers, with an initial working volume of 1.5 L YP1% D [1% (w/v) Bacto yeast extract, 2% (w/v) Bacto peptone, 1% (w/v) dextrose] and 50 mL seed culture.

The bioreactor cultivations were inoculated at pH 6.6. The pH was not controlled throughout the course of the experiment. A 600 g/L glucose solution was used as carbon feed. DO was controlled at 30% saturation by varying agitation from 400 to 600 rpm (cascade mode to control DO in batch phase and fed-batch phase unless stated otherwise), at an aeration rate of 1.5 LPM (1 VVM). Fermentation temperature was held constant at 30° C.

Process values were monitored and recorded using the integrated Sartorius software (BioPAT MFCS/win). Feeding parameters were implemented using customized LabVIEW Virtual Instruments (National Instruments, Austin, Tex.). Exhaust gas oxygen and carbon dioxide compositions were monitored and recorded using BlueSens offgas analyzers (BlueInOne Cell, BlueSens gas sensor GmbH, Herten, Germany).

Glucose starvation conditions were achieved by utilizing a DO-based pulse feeding strategy in which glucose was added on demand upon carbon exhaustion. The pulse parameters for the pulse-feed experiments were as follows: Pulse trigger condition were optimized after 17 h of the cultivation to increase the number of starvation events by reducing the amount of glucose fed per pulse (3 g per pulse to 0.6 g per pulse after 17 h). The pulse trigger conditions were as follows: ΔDO=20%; flow rate; 0.167 mL/min; pulse duration, 30 min (first 17 h of feed phase) and 6 min (until end of fermentation).

Glucose excess conditions were achieved by utilizing a fixed rate pulse feeding strategy which aimed at restoring the initial batch glucose concentration of 10 g/L, followed by periodic glucose pulse additions administering a fixed pulse dosage of 10 mL of glucose feed solution or 6 g glucose per hour (4 g/L/h). It is important to note that we did not observe glucose accumulation larger than 1 g/L in the excess feeding strategy.

### REFERENCES CITED IN EXAMPLE 1

1. Casini A, Chang F-Y, Eluere R, King A M, Young E M, Dudley Q M, et al. A pressure test to make 10 molecules in 90 days: external evaluation of methods to engineer biology. J Am Chem Soc. 2018; 140:4302-16.
2. Enfors S O, Jahic M, Rozkov A, Xu B, Hecker M, Jürgen B, et al. Physiological responses to mixing in large scale bioreactors. J Biotechnol. 2001; 85:175-85.
3. Takors R. Scale-up of microbial processes: impacts, tools and open questions. J Biotechnol. 2012; 160:3-9.
4. Lara A R, Galindo E, Ramirez O T, Palomares L A. Living with heterogeneities in bioreactors: understanding the effects of environmental gradients on cells. Mol Biotechnol. 2006; 34:355-81.
5. Wang G, Huang M, Nielsen J. Exploring the potential of Saccharomyces cerevisiae for biopharmaceutical protein production. Curr Opin Biotechnol. 2017; 48:77-84.
6. Yu T, Zhou Y J, Wenning L, Liu Q, Krivoruchko A, Siewers V, et al. Metabolic engineering of Saccharomyces cerevisiae for production of very long chain fatty acid-derived chemicals. Nat Commun. 2017; 8:15587.
7. Gancedo J M. Yeast carbon catabolite repression. Microbiol Mol Biol Rev. 1998; 62:334-61.
8. De Deken R. The crabtree effect: a regulatory system in yeast. J Gen Microbiol. 1966; 44:149-56.
9. Postma E, Verduyn C, Scheffers W A, Van Dijken J P. Enzymic analysis of the crabtree effect in glucose-limited chemostat cultures of Saccharomyces cerevisiae. Appl Environ Microbiol. 1989; 55:468-77.
10. 10. Frick O, Wittmann C. Characterization of the metabolic shift between oxidative and fermentative growth in Saccharomyces cerevisiae by comparative 13C flux analysis. Microb Cell Fact. 2005; 4:30.
11. DeRisi J L, Iyer V R, Brown P O. Exploring the metabolic and genetic control of gene expression on a genomic scale. Science. 1997; 278:680-6.
12. Zampar G G, Kümmel A, Ewald J, Jol S, Niebel B, Picotti P, et al. Temporal system-level organization of the switch from glycolytic to gluconeogenic operation in yeast. Mol Syst Biol. 2013; 9:651.
13. Roberts G G, Hudson A P. Transcriptome profiling of Saccharomyces cerevisiae during a transition from fermentative to glycerol-based respiratory growth reveals extensive metabolic and structural remodeling. Mol Genet Genomics. 2006; 276:170-86.
14. Fendt S-M, Sauer U. Transcriptional regulation of respiration in yeast metabolizing differently repressive carbon substrates. BMC Syst Biol. 2010; 4:12.
15. Degreif D, de Rond T, Bertl A, Keasling J D, Budin I. Lipid engineering reveals regulatory roles for membrane fluidity in yeast flocculation and oxygen-limited growth. Metab Eng. 2017; 41:46-56.
16. Plata C, Mauricio J C, Millán C, Ortega J M. Influence of glucose and oxygen on the production of ethyl acetate and isoamyl acetate by a Saccharomyces cerevisiae strain during alcoholic fermentation. World J Microbiol Biotechnol. 2005; 21:115-21.
17. Otero J M, Cimini D, Patil K R, Poulsen S G, Olsson L, Nielsen J. Industrial systems biology of Saccharomyces cerevisiae enables novel succinic acid cell factory. PLoS ONE. 2013; 8:e54144.
18. Jouhten P, Rintala E, Huuskonen A, Tamminen A, Toivari M, Wiebe M, et al. Oxygen dependence of metabolic fluxes and energy generation of Saccharomyces cerevisiae CEN.PK113-1A. BMC Syst Biol. 2008; 2:60.
19. Takahashi H, Kumagai T, Kitani K, Mori M, Matoba Y, Sugiyama M. Cloning and characterization of a Streptomyces single module type non-ribosomal peptide synthetase catalyzing a blue pigment synthesis. J Biol Chem. 2007; 282:9073-81.
20. Chavan R B. Environmentally friendly dyes. Handbook of textile and industrial dyeing. Amsterdam: Elsevier; 2011. p. 515-61.
21. Lambalot R H, Gehring A M, Flugel R S, Zuber P, LaCelle M, Marahiel M A, et al. A new enzyme superfamily—the phosphopantetheinyl transferases. Chem Biol. 1996; 3:923-36.
22. Owen J G, Copp J N, Ackerley D F. Rapid and flexible biochemical assays for evaluating 4′-phosphopantetheinyl transferase activity. Biochem J. 2011; 436:709-17.
23. Mutka S C, Bondi S M, Carney J R, Da Silva N A, Kealey J T. Metabolic pathway engineering for complex polyketide biosynthesis in Saccharomyces cerevisiae. FEMS Yeast Res. 2006; 6:40-7.
24. Wimpenny J W, Coombs J P. Penetration of oxygen into bacterial colonies. J Gen

Microbiol. 1983; 129:1239-42.
25. Reverchon S, Rouanet C, Expert D, Nasser W. Characterization of indigoidine biosynthetic genes in Erwinia chrysanthemi and role of this blue pigment in pathogenicity. J Bacteriol. 2002; 184:654-65.
26. Day M. Yeast petites and small colony variants: for everything there is a season. Adv Appl Microbiol. 2013; 85:1-41.
27. Brown C M, Johnson B. Influence of the concentration of glucose and galactose on the physiology of Saccharomyces cerevisiae in continuous culture. J Gen Microbiol. 1970; 64:279-87.
28. Crosby B, Colson A-M, Briquet M, Moustacchi E, Goffeau A. Basic for slow growth on non-fermentable substrates by a Saccharomyces cerevisiae mutant UV-sensitive for rho? production. Mol Gen Genet. 1978; 164:227-34.
29. Fiechter A, Fuhrmann G F, Kappeli O. Regulation of glucose metabolism in growing yeast cells. Adv Microb Physiol. 1981; 22:123-83.
30. Gombert A K, dos Santos M M, Christensen B, Nielsen J. Network identification and flux quantification in the central metabolism of Saccharomyces cerevisiae under different conditions of glucose repression. J Bacteriol. 2001; 183:1441-51.
31. Haushalter R W, Kim W, Chavkin T A, The L, Garber M E, Nhan M, et al. Production of anteiso-branched fatty acids in Escherichia coli; next generation biofuels with improved cold-flow properties. Metab Eng. 2014; 26:111-8.
32. Cann A F, Liao J C. Production of 2-methyl-1-butanol in engineered Escherichia coli. Appl Microbiol Biotechnol. 2008; 81:89-98.
33. Yim H, Haselbeck R, Niu W, Pujol-Baxley C, Burgard A, Boldt J, et al. Metabolic engineering of Escherichia coli for direct production of 1,4-butanediol. Nat Chem Biol. 2011; 7:445-52.
34. Chung H, Yang J E, Ha J Y, Chae T U, Shin J H, Gustaysson M, et al. Bio-based production of monomers and polymers by metabolically engineered microorganisms. Curr Opin Biotechnol. 2015; 36:73-84.
35. Fu Z, Verderame T D, Leighton J M, Sampey B P, Appelbaum E R, Patel P S, et al. Exometabolome analysis reveals hypoxia at the up-scaling of a Saccharomyces cerevisiae high-cell density fed-batch biopharmaceutical process. Microb Cell Fact. 2014; 13:32.
36. McMillan J D, Beckham G T. Thinking big: towards ideal strains and processes for large-scale aerobic biofuels production. Microb Biotechnol. 2017; 10:40-2.
37. Meadows A L, Hawkins K M, Tsegaye Y, Antipov E, Kim Y, Raetz L, et al. Rewriting yeast central carbon metabolism for industrial isoprenoid production. Nature. 2016; 537:694-7.
38. Bylund F, Collet E, Enfors S O, Larsson G. Substrate gradient formation in the large-scale bioreactor lowers cell yield and increases by-product formation. Bioprocess Eng. 1998; 18:171.
39. Larsson G, Törnkvist M, Wernersson E S, Trägåardh C, Noorman H, Enfors S O. Substrate gradients in bioreactors: origin and consequences. Bioprocess Eng. 1996; 14:281-9.
40. Ham T S, Dmytriv Z, Plahar H, Chen J, Hillson N J, Keasling J D. Design, implementation and practice of JBEI-ICE: an open source biological part registry platform and tools. Nucleic Acids Res. 2012; 40:e141.
41. Lee F W F, Silva N A D. Improved efficiency and stability of multiple cloned gene insertions at the δ sequences of Saccharomyces cerevisiae. Appl Microbiol Biotechnol. 1997; 48:339-45.
42. Reider Apel A, d'Espaux L, Wehrs M, Sachs D, Li R A, Tong G J, et al. A Cas9-based toolkit to program gene expression in Saccharomyces cerevisiae. Nucleic Acids Res. 2017; 45:496-508.
43. Gietz R D, Woods R A. Transformation of yeast by lithium acetate/singlestranded carrier DNA/polyethylene glycol method. Guide to yeast genetics and molecular and cell biology—part B. Amsterdam: Elsevier; 2002. p. 87-96.
44. Pfeifer B A, Admiraal S J, Gramajo H, Cane D E, Khosla C. Biosynthesis of complex polyketides in a metabolically engineered strain of E. coli. Science. 2001; 291:1790-2.
45. Schindelin J, Arganda-Carreras I, Frise E, Kaynig V, Longair M, Pietzsch T, et al. Fiji: an open-source platform for biological-image analysis. Nat Methods. 2012; 9:676-82.
46. Yu D, Xu F, Valiente J, Wang S, Zhan J. An indigoidine biosynthetic gene cluster from Streptomyces chromofuscus ATCC 49982 contains an unusual IndB homologue. J Ind Microbiol Biotechnol. 2013; 40:159-68.
47. Kuhn R, Starr M P, Kuhn D A, Bauer H, Knackmuss H J. Indigoidine and other bacterial pigments related to 3,3′-bipyridyl. Arch Mikrobiol. 1965; 51:71-84.
48. Ljungdahl P O, Daignan-Fornier B. Regulation of amino acid, nucleotide, and phosphate metabolism in Saccharomyces cerevisiae. Genetics. 2012; 190:885-929.
49. Chen J, Sutter B M, Shi L, Tu B P. GATOR1 regulates nitrogenic cataplerotic reactions of the mitochondrial TCA cycle. Nat Chem Biol. 2017; 13:1179-86.

### Example 2

### Expanding the Range of Heterologous Products in R. toruloides: Sustainable Bioproduction of the Non-Ribosomal Peptide Indigoidine

Non-ribosomal peptides (NRPs) constitute a diverse class of valuable secondary metabolites, with potential industrial applications including pharmaceuticals, polymers and dyes. However, the low amounts of NRPs produced from native sources and their structural complexity impede mass production by purification from native sources or by chemical synthesis. To extend the range of microbial hosts that can be used for efficient heterologous production of NRPs, we engineered the fungal host Rhodosporidium toruloides to produce the blue NRP pigment indigoidine. Using colorimetric production assays, we demonstrated the ability of R. toruloides to convert various low-cost carbon and nitrogen sources into indigoidine. We established that the colorimetric features of the product are dependent on the pH of the culture as well as the oxidation state of the molecule. To demonstrate production from renewable carbon sources and assess process scalability we produced indigoidine in 2 L bioreactors, reaching titers of 2.9±0.8 g/L using hydrolysates derived from lignocellulosic biomass in a batch process and 18.04±1.50 g/L using a high-gravity fed-batch process. This study represents the first heterologous production of an NRP in R. toruloides. To our knowledge, this is also the first demonstration of production of indigoidine or any NRP using hydrolysates derived from lignocellulosic biomass. These results highlight the potential of R. toruloides for the sustainable and scalable production of NRPs, with the highest reported titer of indigoidine or any heterologously produced NRP to date.

### Introduction

Public awareness of eco-safety and health concerns associated with the production and use of certain toxic compounds used throughout the commercial sector has given rise to a growing demand for environmentally sustainable natural products. Secondary metabolites such as polyketides and non-ribosomal peptides (NRPs) have generated significant interest because of their important roles in a wide range of industries including pharmaceuticals, polymers, flavors and fragrances, and natural dyes.¹⁻³ Natural dyes have been used for centuries to color textiles,⁴ cosmetics, and food ingredients.⁵ Current industrial production of dyes is predominantly achieved via chemical synthesis, which can involve toxic precursors such as petroleum-derived aniline and generate hazardous chemicals as byproducts of the process. Microbial production of these dyes has the potential to address the environmental concerns of harsh chemical synthesis while providing higher production levels and purity than achieved by native sources.^{6,7}

Indigoidine is a natural blue pigment natively produced by several bacteria via biosynthetic gene clusters.⁸⁻¹² This 3′,3′-bipyridyl pigment is formed through condensation of two molecules of L-glutamine catalyzed by a non-ribosomal peptide synthetase (NRPS).^{8,10} NRPS are large assembly-line enzymes and are organized in modules that are each responsible for the introduction of one L-amino acid to the NRP. Each module consists of several domains with defined functions that synthesize NRPs in a sequential multi-step process. The secondary metabolite class of NRPs includes molecules with a range of pharmaceutical applications, such as immunosuppressants, antibiotics, anticancer drugs and antiviral compounds.¹ However, the low NRP production levels from native hosts and their complex chemical structures impede mass production by purification from biological material or chemical synthesis. Furthermore, despite the availability of biosynthetic tools for metabolic engineering and pathway discovery, optimization of NRP production in their natural hosts remains challenging.

The basidiomycete Rhodosporidium toruloides, also known as Rhodotorula toruloides, is emerging as a robust and metabolically flexible host for bioproduction. This oleaginous red yeast natively produces high amounts of lipids, carotenoids and industrially relevant enzymes.¹³ Furthermore, R. toruloides features many host characteristics vital for commercial-scale production, such as the capacity to grow to high cell densities,¹⁴ the ability to utilize a wide range of nitrogen and carbon sources¹⁵ and tolerance to inhibitory compounds found in unrefined substrates.¹⁶ In addition to its native and convenient industrial features, recent development in metabolic engineering tools and -omics techniques for R. toruloides¹⁷⁻²⁰ enabled not only further optimization of native production levels but also the expansion of target molecules produced in this host. While R. toruloides has successfully been engineered to produce heterologous products from pathways that natively have high carbon flux, like fatty acid-derived products²¹ and non-native terpenes,²² R. toruloides has not been explored for the production heterologous NRPs.

In this study, we examine the oleaginous yeast R. toruloides as platform host for heterologous NRP production. For this purpose, we genetically engineered R. toruloides to express the NRPS BpsA from Streptomyces lavendulae enabling the production of the blue pigment indigoidine. Using colorimetric production assays, we analyzed production in different cultivation conditions and at different scales. We show that R. toruloides is capable of converting various low-cost carbon and nitrogen sources to indigoidine and demonstrate the importance of carbon-nitrogen ratio for efficient production. To our knowledge, this is the first report of NRP production in R. toruloides. Our findings emphasize the potential of R. toruloides to serve as a robust platform host for heterologous production of NRPs.

### Experimental

### Strain Construction

The haploid R. toruloides strain IFO0880 served as parental strain for this study. BpsA and sfp sequences were codon-optimized for expression in R. toruloides. Gene synthesis and plasmid construction of pgen335.sfp.bpsA were performed by Genscript (Piscataway N.J.). Pgen335.sfp.bpsA contains a Nourseothricin selection marker. All genes were expressed from native promoters: BpsA was expressed using Tef1p, while Sfp expression was driven by Acp1p. All strains and plasmids used in this study have been deposited in the public instance of the JBEI Registry²³ along with their corresponding information and are available upon request through the Joint BioEnergy Institute Strain Registry (website for: public-registry.jbei.org/folders/419). Pgen335.sfp.bpsA was introduced into R. toruloides recipient strain by Agrobacterium tumefaciens-mediated transformation (ATMT) as previously described.¹⁸

### Media and Cultivation Conditions

Overnight cultures of R. toruloides were grown in 5 mL standard rich glucose medium (YPD, 1% (w/v) Bacto yeast extract, 2% (w/v) Bacto peptone, 2% (w/v) Dextrose) from a single colony at 30° C., shaking at 200 rpm, unless otherwise stated. These overnight cultures were used to inoculate production cultures to OD₈₀₀ of 0.15, which were cultivated at 25° C., shaking at 200 rpm, unless indicated otherwise. All productions were carried out in three or four technical replicates.

All experiments were performed in synthetic defined medium (SD, 1.7 g/L yeast nitrogen base without amino acids and ammonium sulfate (Becton Dickinson, Franklin Lakes N.J.), 0.79 g/L complete supplement mixture without yeast nitrogen base (Sunrise Science Products, San Diego Calif.) with 100 g/L sugar and 10.6 g/L urea as nitrogen base) unless stated otherwise. The synthetic defined media was buffered with 10 mM citrate buffer at pH 5, except for media used in experiments to characterize nitrogen sources and ratio, which was buffered with 10 mM phosphate buffer at pH 6.

The pretreatment configuration of the lignocellulosic feedstocks, Eucalyptus and switchgrass, and the characterization of resulting hydrolysates are described in detail elsewhere²⁴. The hydrolysates were mixed in a 9:1 ratio (v/v) with 10× synthetic defined media (17 g/L yeast nitrogen base without amino acids and ammonium sulfate (Becton Dickinson, Franklin Lakes N.J.), 7.9 g/L complete supplement mixture without yeast nitrogen base (Sunrise Science Products, San Diego Calif.) 79.5 g/L urea as nitrogen base) and buffered with 10 mM citrate buffer at pH 5.

### Indigoidine Extraction

Indigoidine was extracted using a previously developed protocol by Wehrs et al. with slight modifications.²⁵ Briefly, 0.5 mL of culture was centrifuged at 21,000 g for 3 min and the supernatant removed. For cell lysis and simultaneous extraction of indigoidine, 100 μL of acid washed beads (625 nm) and 2 mL DMSO+2% Tween® 20 were added to the cell pellet and vortexed twice for 1 min using Mini-Beadbeater-96 (Biospec, Bartlesville Okla.) at 3600 rpm. After centrifugation at 21,000 g for 3 min, the indigoidine concentration was determined by measuring the OD₆₁₂ of the supernatant using a BioTek Synergy 4 plate reader (Biotek, Winooski Vt.), preheated to 25° C. and applying a standard curve, prepared as described using indigoidine purified from BlueBelle cultures as described in Yu et al.⁹

### Measurement of Absorption Spectra of Indigoidine in Supernatant at Different pH and Oxidation States

To test the effect of varying pH on the pigment, the pH of 500 μL of cell culture supernatant was adapted to indicated values using 1M HCl or 1M NaOH. Subsequently, 100 μL of the supernatant were transferred to a well of a 96-black-well plate. Absorption spectra were measured using a BioTek Synergy 4 plate reader, preheated to 25° C.

### Sugar Quantification

Sugar concentrations were quantified on a 1200 series HPLC (Agilent Technologies) equipped with an Aminex H column (Bio-Rad, Hercules Calif.). To remove cells, samples were filtered through 0.45 μm filters (VWR) and 5 μL of each sample was injected onto the column, preheated to 50° C. The column was eluted with 4 mM sulfuric acid (H₂SO₄) at a flow rate of 600 μL/min for 20 minutes. The eluents were monitored by a refractive index detector, and concentrations were calculated by peak area comparison to known standards.

### Separation-Free Process Coupling Pretreatment, Saccharification and Fermentation

The one-pot process using sorghum biomass was performed according to Sundstrom et al. using the same biomass with minor modifications.¹⁶ Briefly, the ionic liquid pretreatment and enzymatic hydrolysis was performed in a 10 L Parr reactor (Parr Instrument Company, Moline, Ill., USA). Twenty-five percent biomass loading was achieved by using 600 g of biomass in a 10:90 ratio of cholinium lysinate [Ch][Lys] and water. The pretreatment was carried out at 140° C. for 1 h, stirring at 50 rpm using three arm, self-centering anchor with PTFE wiper blades.

Following pretreatment, the reactor was cooled down and the pH was adjusted to pH 5 with 50% v/v H₂SO. The IL-treated biomass was diluted with DI water to achieve a solid loading of 20% w/w. The cellulase complex Cellic® CTec2 and HTec2 (Novozymes, Franklington, N.C., USA) (ratio of 9:1 v/v) was used at loading of 10 mg/g biomass dosing 31.9 mL of the (hemi) cellulolytic enzymatic cocktails (i.e. 53 mL/kg biomass). The reaction vessel was mixed at 30 rpm and heated to 50° C. for 72 h. The amount of sugar generated by hydrolysis was monitored by taking an aliquot from the slurry at specific times and analyzed by HPLC for sugar quantification as described. Enzymatic digestibility was defined as the glucose yield based on the maximum potential glucose from glucan in biomass. In the calculation of cellulose conversion to glucose, it was considered a cellulose: glucose ratio of 1:1.11.²⁶ Prior to use, the hydrolysate was pasteurized at 80° C. for 3 h in a BINDER FED 720 heating chamber (BINDER GmbH, Tuttlingen, Germany).

The subsequent indigoidine production was performed using 2 L Sartorius BIOSTAT B® fermentation system (Sartorius AG., Goettingen, Germany), each agitated with two Rushton impellers, with an initial working volume of 1 L unfiltered sorghum hydrolysate containing 7.95 g/L urea and 50 mL seed culture pre-cultured overnight in a 50:50 mixture of YP10% D (1% (w/v) Bacto yeast extract, 2% (w/v) Bacto peptone, 10% (w/v) Dextrose) and filtered hydrolysate (0.2 μm filter), adjusted to pH 7. The bioreactor cultivations were inoculated at pH 7 and adjusted to maintain below pH 7.5 using HCl throughout the process (initially 1 N HCl, switched to 6 N HCl after 48 h). 1 mL 30% cefotaxime was added to the batch medium to inhibit contamination. Process values were monitored and recorded using the integrated Sartorius software (BioPAT MFCS/win). Dissolved oxygen was controlled to remain above a set point of 30% by varying agitation from 400-900 rpm. Fermentation temperature was held constant at 25° C.

### Fed-Batch Experiments at 2 L Bioreactor Scale

Fed-batch experiments were performed using 2 L Sartorius BIOSTAT fermentation system (Sartorius AG., Goettingen, Germany), each agitated with two Rushton impellers, with an initial working volume of 1 L synthetic defined medium (SD, 1.7 g/L yeast nitrogen base without amino acids and ammonium sulfate (VWR International), 0.79 g/L complete supplement mixture without yeast nitrogen base (Sunrise Science Products) with 100 g/L glucose and 10.6 g/L urea as nitrogen base) and 50 mL seed culture pre-cultured overnight in the same media.

The bioreactor cultivations were inoculated at pH 5. Initially, the pH was not controlled, and pH increased to pH 8 after 40 h. To avoid oxidation of newly produced indigoidine, the pH was then adjusted to pH 7 using HCl (initially 1 N HCl, switched to 6 N HCl after 48 h of cultivation). A 600 g/L glucose solution containing 63.6 g/L urea was used as carbon and nitrogen feed. 1 mL 30% cefotaxime was added to the batch medium to inhibit contamination. Process values were monitored and recorded using the integrated Sartorius software (BioPAT MFCS/win). Feeding parameters were implemented using customized LabVIEW Virtual Instruments (National Instruments, Austin, Tex.).

Dissolved oxygen was controlled to 30% by varying agitation between 400-900 rpm. Cultivation temperature was held constant at 25° C. Batch glucose was allowed to drop to approx. 20 g/L before starting continuous feed addition at 0.063 mL/min (86 h EFT). The feed rate was later modified to 0.189 mL/min (96 h EFT), 0.130 mL/min (116 h EFT), and 0.145 mL/min (125 h EFT) in an attempt to maintain glucose concentrations between 20 g/L and 100 g/L. Feed addition was stopped after measuring glucose concentrations higher than 100 g/L (144 h EFT).

### Indigoidine Purification and Chemical Analysis

Purification of indigoidine was performed using a modified protocol from Yu et al.⁹ To obtain pure indigoidine for further chemical analysis, the culture broth was centrifuged at 10 000 g for 5 min to separate the cells from the media. After removal of the supernatant, the cell pellet was resuspended in an equal volume of dimethylformamide (DMF) and cell lysis was performed by subjecting the cells to an using ultrasonic bath (Model 5510, Branson Ultrasonics, Fremont Calif.) for 30 min to facilitate extraction of indigoidine into the solvent. THF can be used for extraction of indigoidine as an alternative to DMF. The extraction process was repeated with fresh solvent until the supernatant was colorless and the organic solution was combined, evaporated in vacuo, and the resultant dark solid was washed twice each with water, methanol, ethyl acetate and hexane (15 mL×2) using centrifugation. Finally, the product was dried under high vacuum to yield 400 mg dry indigoidine from 400 mL culture broth with a concentration of 1 g L⁻¹ indigoidine. Solution 1H nuclear magnetic resonance (NMR) spectra were recorded on a Bruker AVQ-400 (400 MHz) spectrometer (Billerica, Mass.) operating with an Avance electronics console (Las Vegas, Nev.).

### Results and Discussion

R. toruloides as a Platform Host for Production of the Non-Ribosomal Peptide Indigoidine

Several characteristic biosynthetic gene clusters from various bacteria have been identified to produce the blue pigment indigoidine. In the soil bacterium Streptomyces lavendulae, the native pathway that converts L-glutamine to indigoidine consists of two genes: the single module type NRPS BpsA and a 4′-phosphopantetheinyl transferase (PPTase), required to activate the apo-NRPS into its holo-form via the addition of the phosphopantetheinyl group from Coenzyme A.^{8,27} To establish the indigoidine pathway in R. toruloides, we genomically integrated codon-optimized sequences of the Bacillus subtilis PPTase sfp, previously shown to successfully activate the S. lavendulae apo-BpsA in S. cerevisiae,²⁵ and the single module type NRPS gene bpsA into R. toruloides IFO0880 via Agrobacterium tumefaciens-mediated transformation (ATMT).¹⁹

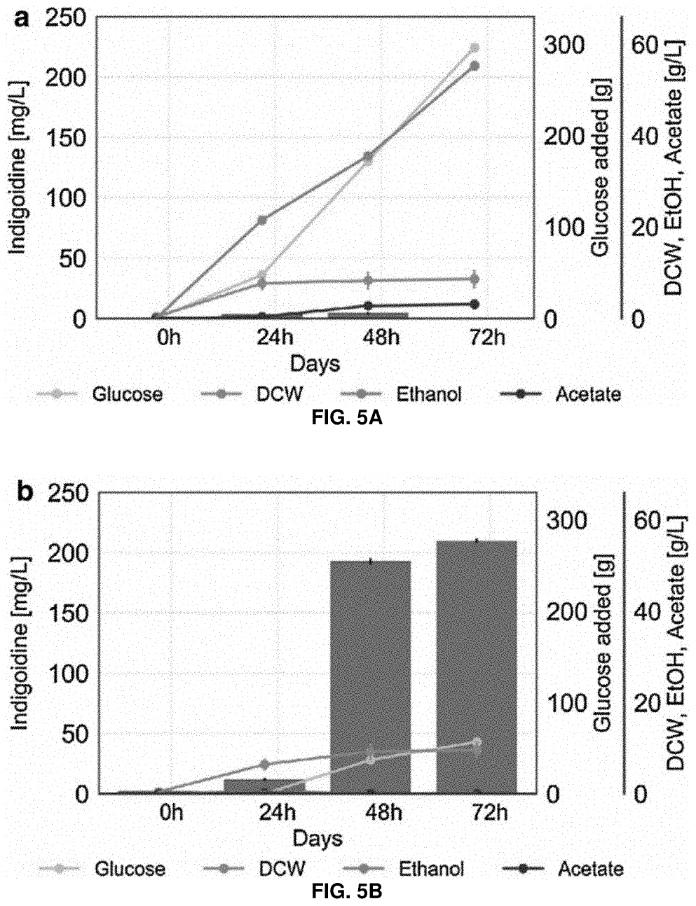

Transformants that successfully expressed these two genes were dark blue due to high-level production of indigoidine. In addition to strong pigmentation of the resultant colonies, the surrounding agar showed dark blue tinting that increased in intensity over time, indicating secretion of indigoidine from the cells. This behavior was maintained in liquid cultures, where the colorization appeared in both the cell pellet and the supernatant (FIG. 7B).

After initial demonstration of indigoidine formation in R. toruloides, we optimized indigoidine production by assessing the impact of physical cultivation parameters on production efficiency. A single transformant (hereafter referred to as BlueBelle) was selected for the optimization. Physical cultivation parameters, including oxygen availability and temperature of the culture, are known to affect the productivity of microbial systems. The filling volume, and correspondingly the headspace of a culture, is recognized to impact oxygen transfer from the gaseous into the liquid phase.²⁸⁻³⁰ Thus, to characterize the effect of oxygen transfer and cultivation temperature on the efficiency of indigoidine production, we compared indigoidine production of BlueBelle cultivated in culture tubes using three different filling volumes (3 mL, 5 mL and 10 mL) after 3 days. Oxygenation is a step required for the formation of indigoidine from glutamine.¹⁰ Hence, we expected the superior oxygen transfer associated with lower filling volume of culture tubes to positively affect pigment production. We detected no significant difference in pigment production for 3 mL and 5 mL cultures. However, as expected, indigoidine production was reduced by 24% in 10 mL cultures compared to 3 mL and 5 mL cultures (FIG. 7C).

The optimal production temperature of a microbial bioprocess can cause a trade-off between optimal growth temperature and optimal temperature for pathway efficiency, the latter often correlating with production of correctly folded proteins. To determine the optimal temperature for indigoidine production and to establish whether the production of indigoidine is linked with microbial growth, we measured microbial growth by means of OD₈₀₀ measurements of 5 mL production cultures of BlueBelle grown at three different temperatures (18° C., 25° C. and 30° C.) and quantified indigoidine production after three days of production (FIG. 7D, E). We observed no significant difference in the growth profile between cultures grown at 25° C. and 30° C., whereas the growth rate was decreased for cultures at 18° C. (FIG. 7D). While all cultures reached a similar final OD₈₀₀ independent of cultivation temperature, cultures grown at 25° C. and 30° C. reached the final OD₈₀₀ after 3 days of cultivation and cultures grown at 18° C. did not reach the final OD₈₀₀ until 5 days after start of the cultivation. Interestingly, the culture pH tracked with microbial growth: the pH of all cultures increased throughout the course of production and reached a similar final value of 8.9. Similar growth and pH profiles were observed for wild-type cultures cultivated under the same conditions.

Overall, we observed that cultivation temperature impacts indigoidine production more strongly than culture volumes for the conditions tested (FIG. 7E). Amongst the tested temperatures, cultivation at 25° C. resulted in the highest indigoidine titer after 3 days. In comparison to cultures grown at 25° C., the indigoidine titer achieved in cultures grown at 30° C. and 18° C. was reduced by 34% and 57%, respectively. Thus, while BlueBelle shows no significant growth impairment when cultivated at 30° C. compared to 25° C., the production of indigoidine is significantly decreased at higher temperatures.

Assuming that the production of indigoidine is directly coupled with biomass at lower temperatures, the differences in indigoidine titer at 18° C. compared to 25° C. could be caused by the differences of biomass accumulation at these temperatures. To test this hypothesis, we quantified indigoidine after 5 days of cultivation when all cultures had reached similar OD₈₀₀. We found that the indigoidine titer produced by cultures grown at 18° C. increased during this period in comparison to cultures grown at higher temperatures and reached similar final titers of indigoidine as that produced by cultures grown at 25° C. These findings indicate that, at lower temperatures, indigoidine production is linked to microbial growth.

### Indigoidine Reveals pH and Redox Dependent Properties

We observed a change in the hue of indigoidine culture over the course of the dye production. While the supernatant of all production cultures started out blue at initial phases of growth, the color changed to green with increasing pH for all cultures, independent of growth temperature, and to yellow at later stages of the production for cultures grown at 25° C. and 30° C. To better characterize the pH dependent colorimetric properties of this compound, we performed pH adjustments of supernatant from a production culture using 1 M sodium hydroxide (NaOH) and 1 M hydrochloric acid (HCl) and recorded the absorption spectra of the resultant solutions. To avoid prior changes in pigment hue due to changes in the culture pH, we utilized the supernatant of a production culture cultivated at 25° C. for 24 hours at neutral pH, as determined by previous growth experiments (FIG. 7D).

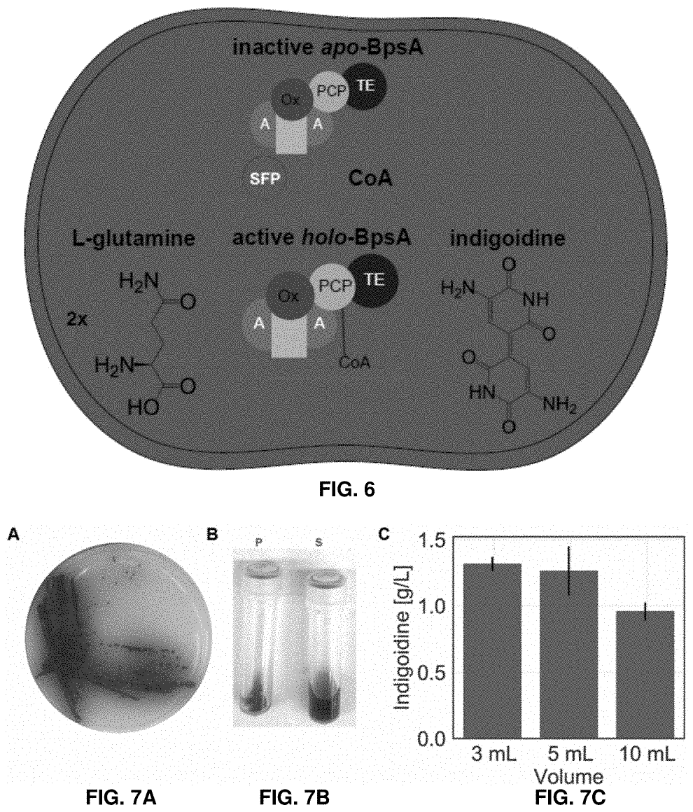

We observed distinct changes in the color of the solution with changes in the pH: the hue of the supernatant ranged from red in acidic conditions to blue at neutral pH, and green under alkaline conditions (FIG. 8). To determine the origin of these colors and confirm the aforementioned observations, we recorded UV-Vis spectra of these solutions with pH ranging from 9 to 2 in intervals of 1. Interestingly, we detected three absorption maxima in these solutions: 470 nm, 500 nm and 610 nm corresponding to perception of orange, red and blue.

The observed increase in red hue with a decrease in the pH was further confirmed by the absorption spectra, where the absorption band at 470 nm becomes more intense with decreasing pH. These observations are in agreement with studies performed by Kuhn et. al to characterize the chemical properties of indigoidine.³¹ During acidification, indigoidine undergoes hydrolysis and the amino functionalities are replaced by hydroxy groups, which changes the chromophore and results in the red pigment, (E)-5,5′-dihydroxy-2H,2′H-[3,3′-bipyridinylidene]-2,2′,6,6′(1H,1′H)-tetraone³¹ (FIG. 8, top left).

To investigate the reversibility of the process, we re-adjusted the pH from pH 2 to pH 7 using 1M NaOH. Indeed, we observed a color change of the solution from red to blue. Kuhn et al determined that the resulting blue color does not originate from the reversion to indigoidine, but rather from the formation of an alkali metal adduct after the addition of the corresponding acid (FIG. 8, bottom left).³¹ However, the process has been observed to be fully reversible when NH₄OH is added as a source for base.³¹ Both the red and blue colors fade over time in the presence of air, resulting in an orange solution with absorption maxima at 470 nm. Similarly, an absorption band at 470 nm was detected in the UV-Vis spectrum of the supernatant at pH 9 that appeared yellow. Thus, we hypothesized the orange pigment to be a product of oxidation. To confirm this hypothesis, we oxidized the various products at different pH (e.g. red pigment at pH 2, blue pigment at pH 7 and orange pigment at pH 9) using hydrogen peroxide. We observed a change in color towards yellow/orange for all three solutions upon addition of the oxidizing reagent, which coincided with the disappearance of the absorption maxima at 610 nm (blue pigment). The orange pigment, identified as [3,3′-bipyridine]-2,2′,5,5′,6,6′(1H,1′H)-hexaone by Kuhn et al.,³¹ was confirmed to be the product of oxidation reactions resulting in absorption maxima at 470 nm (FIG. 8, bottom right). The immediate color change from blue to yellow upon addition of the base NaOH indicates that the oxidation process can be base catalyzed. Addition of the reducing agent dithionite to the various products at different pH (e.g. red pigment at pH 2, blue pigment at pH 7 and orange pigment at pH 9) resulted in loss of color. This observation is in agreement with previous work, describing the formation of a reduced, colorless but fluorescent form, leuco-indigoidine.^{32,33}

Due to the presence of carbon-carbon double bonds conjugated with a carbonyl group in its structure, indigoidine can act as a powerful radical scavenger and strong antioxidant.¹⁰ Cells are constantly exposed to reactive oxygen species (ROS) such as superoxide anion (O₂⁻) or hydrogen peroxide (H₂O₂) of either exogenous or endogenous origin, which can affect their redox balance and lead to oxidative stress.^{34,35} The increased appearance of orange, oxidized pigment in cultures grown at 25° C. and 30° C. in comparison to 18° C., indicates the enhanced presence and release of ROS in cells grown at later stages of growth or at higher temperatures, and highlights the potential use of indigoidine as a redox state sensor.

### Importance of Nitrogen Source for Indigoidine Production

In addition to enhancing physical cultivation parameters like oxygen availability and cultivation temperature, optimization of the production medium composition presents a powerful means to improve a microbial process. The capacity to efficiently utilize multiple, low-cost feedstocks is critical to maintain sustainability and increase economic feasibility of a bioprocess due to potential changes in substrate cost and availability. NRPs in general, and the blue 3′,3′-bipyridyl target molecule indigoidine specifically, are molecules with high nitrogen content, increasing the importance of nitrogen supplied to the culture medium.

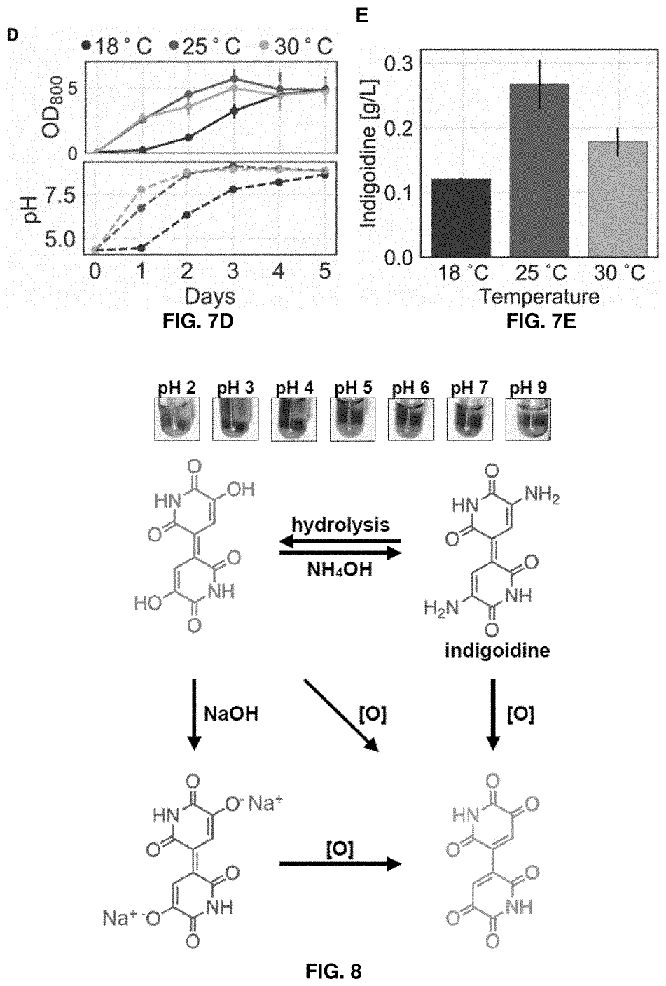

To determine the most suitable nitrogen source for the production of indigoidine in R. toruloides, we cultivated BlueBelle for three days in minimal media containing 100 g/L glucose and individual widely-available nitrogen sources, then quantified indigoidine. To avoid secondary effects originating from carbon-nitrogen ratio, each nitrogen source was normalized to the elemental nitrogen content and added accordingly. In general, indigoidine production varied significantly based on nitrogen source, covering a 4-fold range from the least suitable nitrogen source (potassium nitrate, KNO₃) to the most suitable, which was urea and yeast extract (FIG. 9), emphasizing the importance of the choice of nitrogen source for the production of indigoidine.

The commonly used inorganic nitrogen source ammonium sulfate resulted in comparatively low indigoidine production (FIG. 9). In agreement with studies examining various nitrogen sources for lipid production in R. toruloides,³⁶ this observation indicates the presence of nitrogen catabolite repression (NCR) elicited by extracellular ammonium. The presence of extracellular ammonium has been suggested to repress various catabolic enzymes required for nitrogen assimilation, including glutamate dehydrogenase which catalyzes the reversible reaction of glutamate to alpha-ketoglutarate,³⁶ and this could lead to reduced flux towards the final product, indigoidine.

Other studies have shown that R. toruloides can readily assimilate amino acids as nitrogen source for lipid production under nitrogen-limited conditions.³⁷ To allow utilization of nitrogen for cellular metabolism, any nitrogen source first has to be converted to glutamate or glutamine, which serve as nitrogen donors for all other nitrogen containing compounds in the cell.³⁸ Interestingly, exogenous addition of the amino acids glutamine or glutamate, precursors tin the pathway to indigoidine, did not yield high production of indigoidine (FIG. 9). This observation could originate from slow assimilation or a regulatory response that reduces native flux toward these compounds under non-nitrogen limited conditions. These conclusions are in general agreement with differential transcriptomic and proteomic analyses of R. toruloides under different nitrogen regimes, illustrating the effect of nitrogen availability on central carbon metabolism and lipid metabolism.³⁹

In contrast to the amino acids glutamine and glutamate, use of urea as a nitrogen source resulted in a high titer of indigoidine, indicating that the use of urea as sole nitrogen source elicits faster uptake and assimilation. This conclusion is in agreement with previous reports describing faster uptake of urea and a faster metabolic response as compared to ammonium or glutamate, partly due to upregulation of urease activity when urea is used as nitrogen source.³⁷ Additionally, the enhanced production of indigoidine could result from an increased accumulation of the precursor glutamine. To use urea as a nitrogen donor, the compound is first hydrolyzed by a urease or decarboxylated by an urea amidolyase to yield ammonia, which in turn is used as substrate to form glutamate and glutamine from alpha-ketoglutarate.^{39,40} Indeed, early biochemical studies revealed that enzymes required for L-glutamine synthesis from ammonia show higher activities when urea was used as nitrogen source compared to glutamine.³⁷

The use of yeast extract as a nitrogen source resulted in similarly high titers of indigoidine (FIG. 9). Yeast extract is a complex nitrogen source, predominantly prepared by autolysis of yeast cells, and thus contains all soluble cell compounds, such as peptide and amino acids, vitamins, trace elements and nucleic acids.⁴¹ However, the poorly defined composition, batch-to-batch variability and high cost of commercially available yeast extracts present constraints for industrial processes.^{41,42} Based on these factors, we optimized the process using urea as the defined nitrogen source.

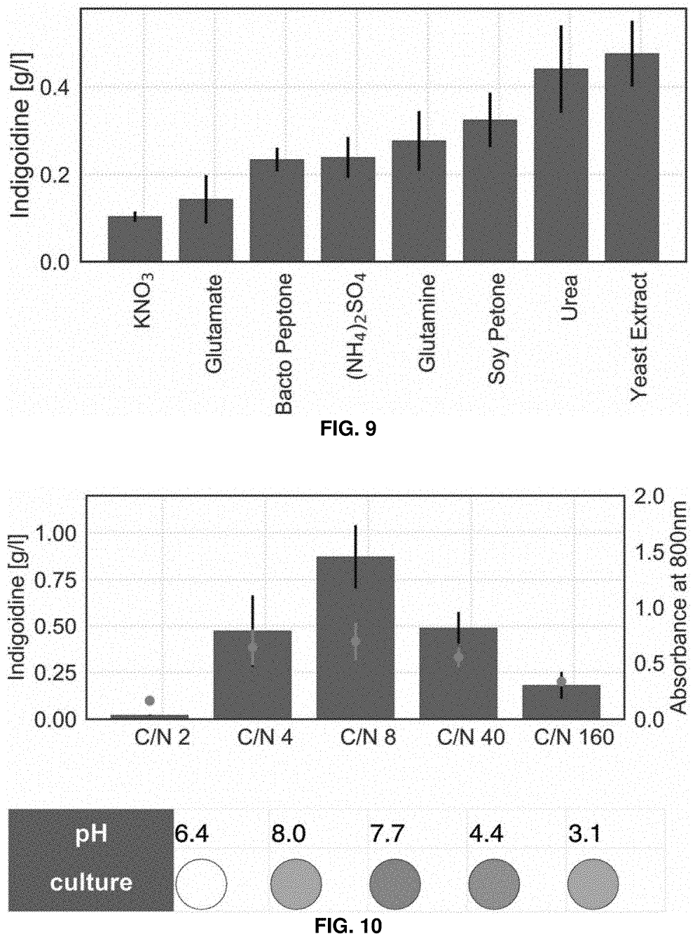

Nitrogen limitation is known to enhance accumulation of storage lipids in oleaginous yeasts.³⁶ The effects of varying nitrogen concentrations and nitrogen limitation on production of lipids and fatty acid-derived products have been thoroughly studied in R. toruloides.⁴³ To better characterize the effect of nitrogen availability on the production of the TCA cycle derived, heterologous product indigoidine, we cultivated BlueBelle in minimal medium containing 100 g/L glucose at different carbon/nitrogen (C/N) ratios, ranging from C/N 2 to C/N 160, and determined indigoidine titer as well as culture pH after 3 days (FIG. 10).

As the C/N ratio presents an important factor known to affect carbon flux distribution into either storage lipids or by-products in oleaginous yeast,⁴⁴⁻⁴⁶ specifically citrate in R. toruloides,³⁹ we expected to find an interdependency between C/N ratio and indigoidine production. Indeed, amongst the conditions tested, we found a C/N ratio of 8 to result in the highest indigoidine production after 3 days. A deviation from this ratio to either lower or higher C/N ratio, e.g. C/N 4 or C/N 40 respectively, resulted in lower titers. Growth was mostly stalled at the lowest C/N ratio tested (C/N 2) as well as the highest C/N ratio tested (C/N 160), possibly due to inhibitory effects originating from high amounts of urea added to the medium (42.5 g/L)⁴⁷ and nitrogen limitation, respectively.

Interestingly, the color of the culture also changed with varying C/N ratio (FIG. 10), indicating changes in pH during the cultivation triggered by carbon or nitrogen limitations. This observation was further confirmed by pH measurements. After 3 days of cultivation, the cultures in low C/N ratio, thus not nitrogen limited, showed a green or blue hue, corresponding to pH values of 8.0 for C/N 4 and 7.7 for C/N 8, respectively. In contrast, cultures grown in media containing a high C/N ratio, thus under nitrogen limitation, showed a purple to red hue, indicating a low pH, which was confirmed by pH measurements. This observation could originate directly from the varying concentrations of urea in these solutions⁴⁸ or from modified metabolic activities. To test whether the increase in pH in cultures containing a low C/N ratio is solely a result of high urea concentrations, we cultivated BlueBelle in rich media with the same concentrations of the complex nitrogen sources yeast extract and peptone containing either 20 g/L (YPD) or 100 g/L glucose (YP10D). Here, as well, we found that the pH increased over time for media having a lower C/N ratio (YPD). This observation renders the hypothesis that the varying concentrations of urea are the sole cause for the differences in culture pH unlikely. However, the observed decrease in culture pH with increasing C/N ratio together with the decrease in production, is consistent with the current understanding of the regulation of lipid accumulation in R. toruloides^{39,43,49} Generally, nitrogen limitation was found to enhance lipid accumulation in oleaginous as well as non-oleaginous yeasts.⁵⁰ Under these conditions, oleaginous yeasts produce large amounts of TCA cycle intermediates, foremost citrate.^{36,51} During nitrogen starvation the activity of the isocitrate dehydrogenase is decreased, which is caused by low levels of its allosteric regulator AMP, resulting from an increased activity of AMP deaminase to ensure a stable pool of internal ammonium required for cell viability.^{39,52} Consequently, mitochondrial citrate is not further metabolized via the TCA cycle resulting in accumulation and subsequent secretion into the cytosol where it serves as substrate to ATP citrate lyase to form acetyl-CoA for fatty acid biosynthesis.^{52,53} Thus, the decrease in production together with the decrease in culture pH with increasing C/N ratio likely originates from the metabolic flux shift to lipid biosynthesis. This shift results in the stalling of flux through the TCA cycle towards the precursor of indigoidine, alpha-ketoglutarate, and is accompanied by enhanced secretion of citrate.³⁷

Further, we observed a delayed increase in indigoidine production after 7 days of cultivation at C/N 40, possibly due to remobilization of storage lipids accumulated during earlier stages of the cultivation as a result of the onset carbon depletion at late stages of the cultivation.^{54,55} This observation further supports the hypothesis, that higher C/N ratios favor storage lipid accumulation over completion of the TCA cycle and underlines the importance of a customized carbon-to-nitrogen ratio for efficient production of different products. Further experiments are needed to fully understand the regulatory mechanisms underlying the balance between lipid biogenesis and TCA cycle progression.

### Utilization of Various Carbon Sources for Indigoidine Production

Various carbon sources, such as glucose, glycerol and biomass derived sugars can feed into the TCA cycle of R. toruloides via different yet integrated pathways, and can be used to produce native products such as lipids and carotenoids as well as heterologous bioproducts with relatively high initial titers such as fatty alcohols and terpenes, all of which funnel mainly through the metabolite acetyl-CoA.^{22,56,57 58} It remains unknown whether TCA cycle intermediates can be used to produce heterologous bioproducts with similar efficiency to the natural products without engineering the carbon flux through central metabolism. Thus, we sought to characterize the ability of R. toruloides to efficiently convert four commonly-used carbon sources into indigoidine: glucose, xylose, sucrose and glycerol.

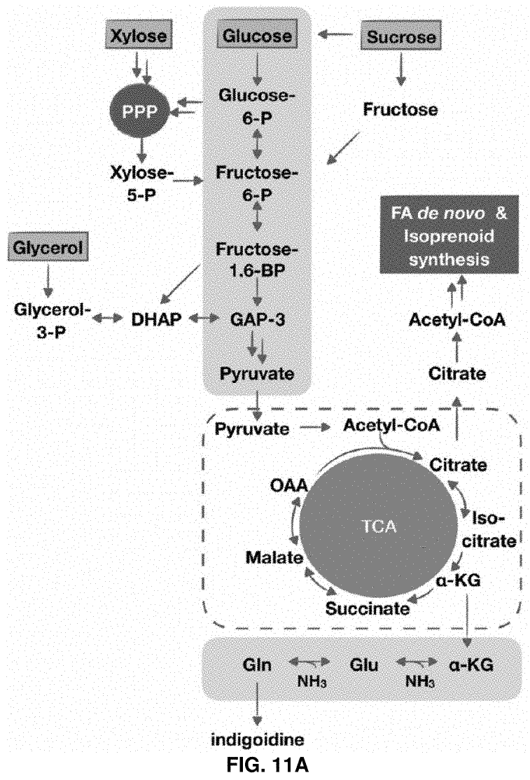

To gain a more detailed understanding of the efficiency of the conversion of these carbon sources, we examined sugar utilization, cell growth, culture pH, and indigoidine production over the course of seven days. Generally, we found that indigoidine production correlated linearly with biomass formation for all the carbon sources tested (FIGS. 11A and 11B). Similar to observations made in the experiment investigating indigoidine production at different cultivation temperatures (FIG. 7D), we found the culture pH to increase with biomass density over the course of the cultivation, reaching similar final values (pH ˜9) in all conditions tested.

Using glucose and sucrose as carbon sources resulted in similar production profiles, especially during the first 3 days of cultivation (FIG. 11B). During this initial phase of production, biomass formation coincided with sugar consumption and indigoidine production. While sugar consumption rate remained comparable for these carbon sources and both sucrose and glucose were fully consumed after 4 days, the growth profiles show differences in the later stages of production (after 4 days). The use of sucrose as carbon source resulted in a further increase in biomass formation from day 3 to day 4, reaching a maximum cell density (OD₈₀₀) of 7.1 on day 4, whereas growth stalled after 3 days when grown on glucose. Interestingly, in both cases, indigoidine titers decreased in the later stages of production.

This observation could be a result of formation of ROS and enhanced metabolic redox associated with the formation of indigoidine from L-glutamine requiring ATP and a flavin cofactor,^{8,59} that lead to the formation of the orange oxidation product of indigoidine, thus limiting the pool of detectable blue indigoidine upon extraction. Further, cultivation in media with a low C/N ratio inhibited the formation of storage lipid,⁶⁰ thus the onset of carbon depletion in the medium could result in starvation responses and additional redox imbalance.⁶¹

Unlike sucrose and glucose, we observed a delay in consumption of glycerol and xylose. This observation indicates the need of adaptation to allow utilization of these carbon sources. For glycerol, this observation is consistent with reports describing carbon catabolite repression after cultivation in glucose containing media (FIG. 11B, right panel).⁵⁸ With the exception of the initial adaptation phase needed for glycerol, the production profile of indigoidine on glycerol as a carbon source is highly similar to the production on glucose and sucrose. Similarly, biomass formation coincided with sugar consumption and indigoidine production, reaching a lower maximum indigoidine titer of 0.7 g/L in glycerol compared to 0.8 g/L in glucose. However, biomass accumulation in glycerol was higher compared to growth on glucose (OD₈₀₀ of 6.21 compared to 5.64). These results are consistent with previous reports⁶² that compared growth and lipid production of R. toruloides on glycerol and glucose.

Of all the carbon sources tested, xylose resulted in the lowest growth and indigoidine titer. Similarly to growth on glycerol, xylose was steadily consumed after an initial adaptation phase, however at a slower rate than any of the other carbon sources as previously reported.²² Xylose is metabolized via the pentose-phosphate-pathway, which results in loss of carbon through CO₂ and thus less biomass and product formation.⁵⁸

Production of the NRP Indigoidine from Lignocellulosic Biomass

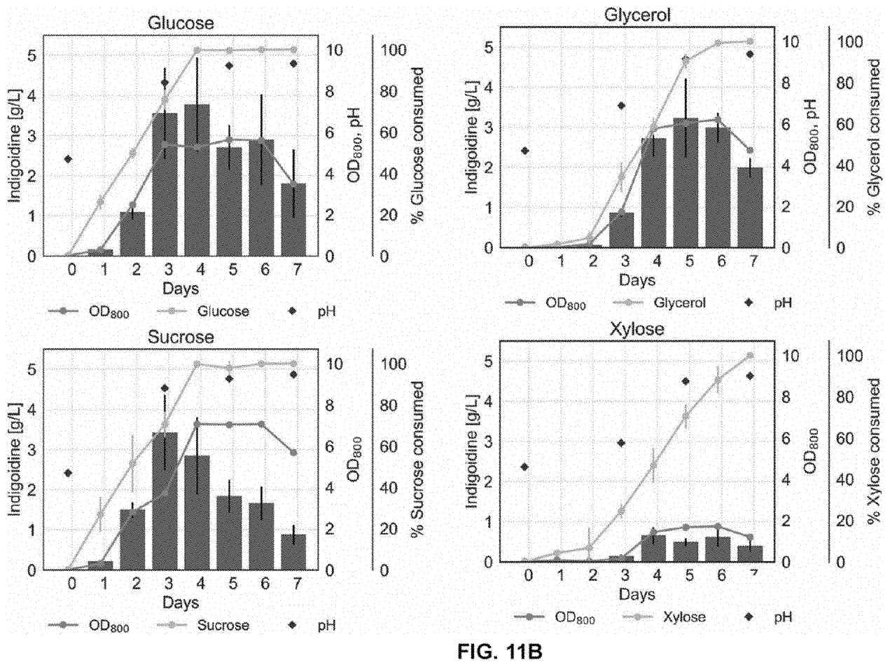

The ability to convert various carbon sources from lignocellulosic biomass to a bioproduct is an important requirement for a sustainable bioprocess from renewable feedstocks. To demonstrate the potential of R. toruloides to produce indigoidine from a mix of carbon liberated from lignocellulosic biomass, we cultivated BlueBelle in hydrolysates derived from several lignocellulosic feedstocks as a sole carbon source. For this purpose, we examined growth, sugar utilization, indigoidine production, and culture pH using hydrolysates from Eucalyptus as well as from mixed feedstocks (switchgrass and Eucalyptus) predominantly containing glucose and xylose (FIG. 12), which were thoroughly characterized in an earlier study.²⁴

In the hydrolysate cultivations, efficient xylose consumption was delayed by 2 days of cultivation, indicative of a required adaptation phase for efficient utilization of xylose as observed when using xylose as sole carbon source for indigoidine production (FIG. 11B, right). Similar to growth on single carbon sources, we found that the indigoidine titer corresponded to biomass accumulation, reaching maximum titers of 0.3 g/L and 0.15 g/L for Eucalyptus and mixed feedstocks, respectively. In contrast to growth on single carbon sources, the maximum indigoidine titer on hydrolysates was reached earlier, after 2 days of growth for both liquors tested, and decreased afterwards, when the culture was not yet depleted of all carbon sources. The early decrease in detectable indigoidine when grown on biomass-derived carbon sources compared to a refined, single carbon source could result from the presence of inhibitors originating from the lignocellulosic biomass.⁶³ This hypothesis is supported by the observation of marked differences in growth and sugar consumption between the two hydrolysates despite having very similar amounts of sugars and total carbon, as different biomass was used for the preparation while maintaining the same deconstruction and saccharification protocol.²⁴

Considering the lower amount of total sugar and specifically lower concentrations of glucose in the hydrolysate obtained from mixed feedstocks and Eucalyptus (FIG. 12) as compared to the 100 g/L of sugar used in the single carbon cultivations, the lower titers obtained from the lignocellulosic hydrolysates are not surprising. Overall, successful production of indigoidine highlights the potential of R. toruloides to convert lignocellulosic biomass and presents the first report of non-ribosomal peptide production from renewable feedstocks.

The use of lignocellulosic hydrolysates as carbon source in many cases includes a solid-liquid separation and several washing steps after the pretreatment process,⁶⁴ which results in loss of lignocellulosic biomass and increased process cost. To demonstrate the feasibility of indigoidine production in R. toruloides from lignocellulosic hydrolysates in a more sustainable manner, we performed a separation-free biomass deconstruction and conversion process using sorghum that couples pretreatment, saccharification and indigoidine production in a single unit operation. R. toruloides was cultivated under this regime in a 2 L bioreactor as previously described by Sundstrom et al. for the production of the terpene bioproduct bisabolene.¹⁶ HPLC analysis showed that the prepared hydrolysate contained 44.3 g/L glucose and 13.8 g/L xylose. To avoid additional cellular stress due to high pH as well as oxidation of the pigment in the culture broth as observed in shake flasks, the pH of the culture was maintained at 7. In order to allow R. toruloides to adapt to the ionic liquid cholinium lysinate used for pretreatment and any other inhibitors present in the hydrolysate, the seed culture used to inoculate the bioreactor contained a 1:1 mix of hydrolysate and rich medium.

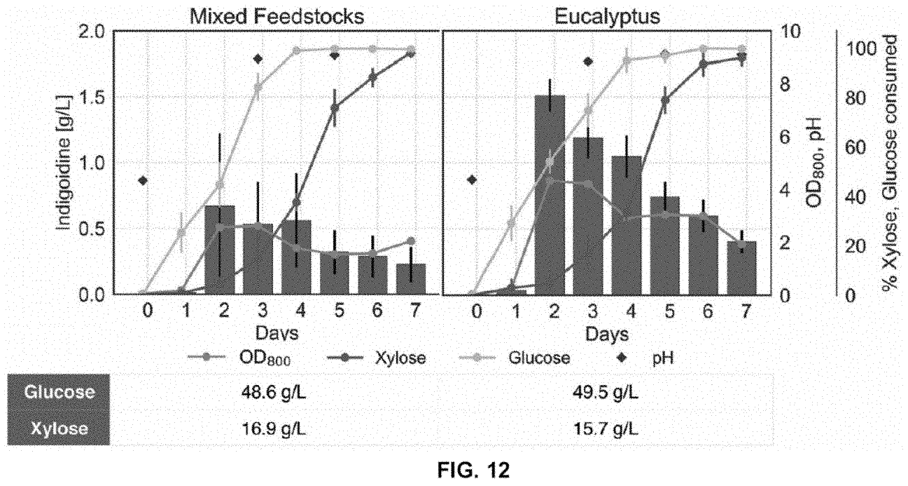

Unlike previous experiments performed in shake flasks, glucose utilization was delayed by a day when grown in the bioreactor using the one-pot lignocellulosic hydrolysate (FIG. 13). This observation suggests a need to adapt to the one-pot environment, including lignin-derived aromatic compounds, some of which can be inhibitory.⁶³ Indeed, Yaegashi et al found that R. toruloides engineered for bisabolene production preferably utilized the aromatic compound p-coumaric acid in a mock medium also containing glucose and xylose.²² Further, they showed that other lignin-related aromatic compounds can be readily consumed by R. toruloides including ferulic acid, even though some were inhibitory.²² Additionally, the ionic liquid used for pretreatment, cholinium lysinate, is known to inhibit microbial growth at higher concentrations. As the seed culture was pre-grown in diluted hydrolysate, the delay in growth observed after transfer to the bioreactor could have been caused by the need for an adaptation phase to the higher concentration of ionic liquid present in the hydrolysate.

After an initial lag, glucose was mostly consumed within 3 days of cultivation. Similar to observations made in previous experiments, efficient xylose consumption was delayed until the majority of glucose was consumed. Indigoidine production commenced with almost complete consumption of glucose at day 3 of the cultivation and increased to a final titer of 2.91 g/L. Overall, the indigoidine yield from sugar achieved in the separation-free bioreactor process was 0.045 g/g, which was 18% higher than the one achieved at shake flask level using the best single carbon source glucose (0.038 g/g) and 95% higher compared to using the best hydrolysate derived from Eucalyptus (0.023 g/g).

Fed-Batch Process with pH Control Results in Increase of Indigoidine Titer

Following cultivation optimization in shake flasks and demonstration of sustainable indigoidine production using lignocellulosic feedstocks, we sought to further enhance indigoidine production in R. toruloides and to demonstrate the feasibility of process scale-up. In previous experiments, we demonstrated a positive correlation between biomass accumulation and indigoidine production. Further, we established the importance of the C/N ratio for efficient indigoidine production and pH for optimal growth of R. toruloides. Thus, we hypothesized that maintaining a neutral pH and ensuring an excess of carbon and nitrogen at an adequate ratio would allow for higher accumulation of biomass while maintaining efficient indigoidine production. To test this hypothesis, we performed a fed-batch cultivation in a 2 L bioreactor at pH 7.

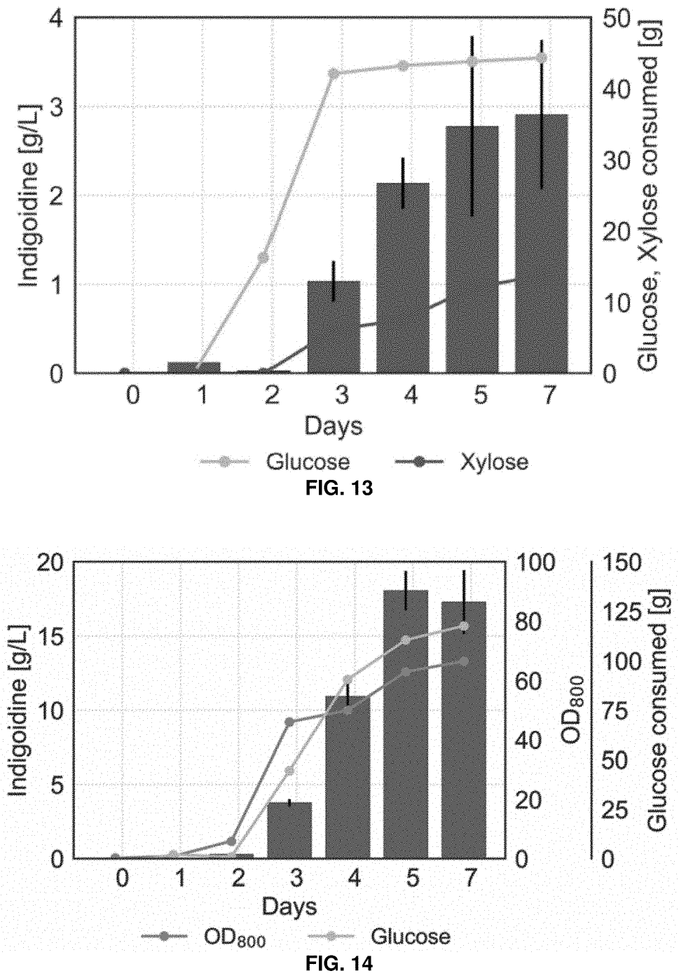

Similar to the experiments performed in shake flasks, indigoidine production tracked with biomass accumulation under these conditions (FIG. 14). However, unlike experiments performed in shake flasks, efficient glucose consumption was delayed by 48 h, indicating the need to adapt to the bioreactor environment. After an initial lag phase with slow glucose consumption and growth, the carbon source was utilized rapidly in the next cultivation phase accompanied by an increase in indigoidine production and biomass formation (days 2 to 4). In the last phase of the cultivation, after 4 days, indigoidine production increased further, while microbial growth slowed down significantly. A maximum indigoidine titer of 18.04±1.50 g/L was achieved after 5 days (116 h) of cultivation with an overall productivity and yield of 0.15 g/L·h and g_{indigoidine}/g_{glucose}, respectively (20.7 g indigoidine net produced and 109.6 g glucose net consumed). To our knowledge, these results represent the highest reported production of NRPs in the scientific literature. The culture reached a final OD₈₀₀ of 66, corresponding to a dry cell weight (DCW) of 27.6±4.9 g/l. The obtained DCW values are similar to those previously reported for high-gravity fed-batch cultivation for bisabolene production using R. toruloides (25 g/L after 135 h of production).²² However, they remain below the DCW values reported for lipid overproduction (106.5 g/L after 134 h of production), likely due the high lipid content accounting for 67.5% g_{lipid}/g_{biomass} or 71.9 g/L of DCW.¹⁴

Our results highlight the potential of R. toruloides for high-carbon fed-batch fermentations, an important aspect when considering microorganisms for industrial applications. In comparison to previous reports of heterologous indigoidine production in the model organisms Escherichia coli at (8.81±0.21 g/l in shake flasks)⁶⁵ and Saccharomyces cerevisiae (209.9 mg/L in 2 L bioreactor),²⁵ the indigoidine titer of 18.04±1.50 g/L achieved in R. toruloides represents a 10 fold and 87 fold increase, respectively.

### Conclusions

Our findings demonstrate the feasibility to employ the basidiomycete yeast R. toruloides to serve as a platform host for heterologous NRP production. Specifically, we investigated the production of the NRP indigoidine, a redox-active blue pigment that is linked to the TCA cycle via its precursor alpha-ketoglutarate. In addition to achieving a high titer of 18.04±1.50 g/L and yield 0.19 g_{indigoidine}/g_{glucose} in a 2 L fed-batch process from glucose and urea, we showed that R. toruloides can convert lignocellulosic hydrolysates and various other carbon and nitrogen sources to indigoidine, and that the efficiency of indigoidine production is partly dependent on the carbon-to-nitrogen ratio.

In addition to containing the same chromophore as the industrially dominant blue dye indigo, indigoidine can act as powerful radical scavenger and strong antioxidant.¹⁰ Further, indigoidine may have potential use as a colorimetric redox state and pH sensor, rendering it a natural blue dye with various industrially important properties. Due to the structural similarity to indigo in terms of amine functional groups that result in strong intermolecular hydrogen bonds and pi-pi stacking interactions, indigoidine may be suitable for similar industrial applications demonstrated for indigo and its derivatives, including biodegradable organic semiconductors and transistors.⁶⁶⁻⁶⁸ However, the nitrogen content of indigoidine is higher compared to indigo, potentially resulting in various different chemical and physical properties, expanding the application space of this class of compounds.

While indigoidine itself shows immense potential for industrial applications, the high titers achieved in this study further underline the potential to explore the basidiomycete R. toruloides as a production host for other classes of heterologous products besides NRPs. The successful demonstration of activating a heterologous NRPS via phosphopantetheinylation suggests that this yeast has potential to produce other classes of compounds that require this step, such as polyketides (PK) or NRP-PK hybrids.⁶⁹

Overall, our study presents the first demonstration of the microbial production of a heterologous NRPS-derived product, the blue pigment indigoidine, at titers approaching 100 g/L. These results suggest that R. toruloides may be key to enabling industrial production of a wide range of valuable NRP and PK bioproducts. Further, our results demonstrate the ability of R. toruloides to facilitate the critical phosphopantetheinylation step required to access to broad category of compounds derived from NRPs and other secondary metabolites. Our results along with the emergence of effective synthetic biology tools and its native industrial features emphasize the potential of R. toruloides as highly versatile microbial production host.

### REFERENCES CITED IN EXAMPLE 2

1. M. A. Martinez-Núñez and V. E. L. y Lopez, Sustain. Chem. Process., 2016, 4, 13.
2. S. Yuzawa, J. D. Keasling, and L. Katz, J Antibiot, 2017, 70, 378-385.
3. P. S. Vankar, Reson., 2000, 5, 73-80.
4. S. Kadolph, Delta Kappa Gamma Bulletin, 2008.
5. A. C. Dweck, Int J Cosmet Sci, 2002, 24, 287-302.
6. T. M. Hsu, D. H. Welner, Z. N. Russ, B. Cervantes, R. L. Prathuri, P. D. Adams, and J. E. Dueber, Nat. Chem. Biol., 2018, 14, 256-261.
7. R. J. N. Frandsen, P. Khorsand-Jamal, K. T. Kongstad, M. Nafisi, R. M. Kannangara, D. Staerk, F. T. Okkels, K. Binderup, B. Madsen, B. L. Møller, U. Thrane, and U. H. Mortensen, Sci. Rep., 2018, 8, 12853.
8. H. Takahashi, T. Kumagai, K. Kitani, M. Mori, Y. Matoba, and M. Sugiyama, J Biol. Chem., 2007, 282, 9073-9081.
9. D. Yu, F. Xu, J. Valiente, S. Wang, and J. Zhan, J. Ind. Microbiol. Biotechnol., 2013, 40, 159-168.
10. S. Reverchon, C. Rouanet, D. Expert, and W. Nasser, J. Bacteriol., 2002, 184, 654-665.
11. R. Kuhn, M. P. Starr, D. A. Kuhn, H. Bauer, and H. J. Knackmuss, Arch. Mikrobiol., 1965, 51, 71-84.
12. M. P. Starr, G. Cosens, and H. J. Knackmuss, Appl Microbiol, 1966, 14, 870-872.
13. Y.-K. Park, J.-M. Nicaud, and R. Ledesma-Amaro, Trends Biotechnol., 2018, 36, 304-317.
14. Y. Li, Z. (Kent) Zhao, and F. Bai, Enzyme Microb. Technol., 2007, 41, 312-317.
15. J. Xu, Journal of Economic Dynamics and Control, 2017, 80, 54-74.
16. E. Sundstrom, J. Yaegashi, J. Yan, F. Masson, G. Papa, A. Rodriguez, M. Mirsiaghi, L. Liang, Q. He, D. Tanjore, T. R. Pray, S. Singh, B. Simmons, N. Sun, J. Magnuson, and J. Gladden, Green Chem., 2018, 20, 2870-2879.
17. C. M. J. Koh, Y. Liu, Moehninsi, M. Du, and L. Ji, BMC Microbiol., 2014, 14, 50.
18. S. Zhang, J. M. Skerker, C. D. Rutter, M. J. Maurer, A. P. Arkin, and C. V. Rao, Biotechnol. Bioeng., 2016, 113, 1056-1066.
19. X. Lin, Y. Wang, S. Zhang, Z. Zhu, Y. J. Zhou, F. Yang, W. Sun, X. Wang, and Z. K. Zhao, FEMS Yeast Res, 2014, 14, 547-555.
20. A. M. B. Johns, J. Love, and S. J. Ayes, Front. Microbiol., 2016, 7, 1666.
21. S. Fillet, C. Ronchel, C. Callejo, M.-J. Fajardo, H. Moralejo, and J. L. Adrio, Appl. Microbiol. Biotechnol., 2017, 101, 7271-7280.
22. J. Yaegashi, J. Kirby, M. Ito, J. Sun, T. Dutta, M. Mirsiaghi, E. R. Sundstrom, A. Rodriguez, E. Baidoo, D. Tanjore, T. Pray, K. Sale, S. Singh, J. D. Keasling, B. A. Simmons, S. W. Singer, J. K. Magnuson, A. P. Arkin, J. M. Skerker, and J. M. Gladden, Biotechnol Biofuels, 2017, 10, 241.
23. T. S. Ham, Z. Dmytriv, H. Plahar, J. Chen, N. J. Hillson, and J. D. Keasling, Nucleic Acids Res., 2012, 40, e141.
24. C. Li, D. Tanjore, W. He, J. Wong, J. L. Gardner, V. S. Thompson, N. A. Yancey, K. L. Sale, B. A. Simmons, and S. Singh, Bioenerg. Res., 2015, 8, 982-991.
25. M. Wehrs, J.-P. Prahl, J. Moon, Y. Li, D. Tanjore, J. D. Keasling, T. Pray, and A. Mukhopadhyay, Microb. Cell Fact., 2018, 17, 193.
26. C. Li, B. Knierim, C. Manisseri, R. Arora, H. V. Scheller, M. Auer, K. P. Vogel, B. A. Simmons, and S. Singh, Bioresour. Technol., 2010, 101, 4900-4906.
27. R. H. Lambalot, A. M. Gehring, R. S. Flugel, P. Zuber, M. LaCelle, M. A. Marahiel, R. Reid, C. Khosla, and C. T. Walsh, Chem. Biol., 1996, 3, 923-936.
28. C. E. Gómez Sánchez, A. Martinez-Trujillo, and G. Aguilar Osorio, Lett. Appl. Microbiol., 2012, 55, 444-452.
29. H. J. Henzler and M. Schedel, Bioprocess Engineering, 1991, 7, 123-131.
30. W. Klöckner and J. Büchs, Trends Biotechnol., 2012, 30, 307-314.
31. R. Kuhn, H. Bauer, and H.-J. Knackmuss, Chem. Ber., 1965, 98, 2139-2153.
32. M. Müllner, S. Ausländer, D. Auslander, C. Kemmer, and M. Fussenegger, Metab. Eng., 2012, 14, 325-335.
33. W. Heumann, D. Young, and C. Gottlich, Biochimica et Biophysica Acta (BBA)—General Subjects, 1968, 156, 429-431.
34. O. N. Oktyabrsky and G. V. Smirnova, Biochemistry Moscow, 2007, 72, 132-145.
35. A. Ayer, C. W. Gourlay, and I. W. Dawes, FEMS Yeast Res, 2014, 14, 60-72.
36. C. T. Evans and C. Ratledge, MICROBIOLOGY, 1984, 130, 1693-1704.
37. C. T. Evans and C. Ratledge, MICROBIOLOGY, 1984, 130, 1705-1710.
38. E. G. ter Schure, N. A. W. van Riel, and C. T. Verrips, FEMS Microbiol. Rev., 2000, 24, 67-83
39. Z. Zhu, S. Zhang, H. Liu, H. Shen, X. Lin, F. Yang, Y. J. Zhou, G. Jin, M. Ye, H. Zou, and Z. K. Zhao, Nat. Commun., 2012, 3, 1112.
40. M. T. Castañeda, S. Nuñez, F. Garelli, C. Voget, and H. De Battista, J. Biotechnol., 2018, 280, 11-18.
41. S. Klotz, A. Kuenz, and U. PruBe, Green Chem., 2017, 19, 4633-4641.
42. J. Zhang, J. Reddy, B. Buckland, and R. Greasham, Biotechnol. Bioeng., 2003, 82, 640-652.
43. H. Liu, X. Zhao, F. Wang, Y. Li, X. Jiang, M. Ye, Z. K. Zhao, and H. Zou, Yeast, 2009, 26, 553-566.
44. M. Tai and G. Stephanopoulos, Metab. Eng., 2013, 15, 1-9.
45. A. Beopoulos, J. Cescut, R. Haddouche, J.-L. Uribelarrea, C. Molina-Jouve, and J.-M. Nicaud, Prog. Lipid Res., 2009, 48, 375-387.
46. S. Papanikolaou and G. Aggelis, Lipid Technol., 2009, 21, 83-87.
47. M. Brabender, M. S. Hussain, G. Rodriguez, and M. A. Blenner, Appl. Microbiol. Biotechnol., 2018, 102, 2313-2322.
48. H. B. Bull, K. Breese, G. L. Ferguson, and C. A. Swenson, Arch. Biochem. Biophys., 1964, 104, 297-304.
49. H. Shen, X. Zhang, Z. Gong, Y. Wang, X. Yu, X. Yang, and Z. K. Zhao, Appl. Microbiol. Biotechnol., 2017, 101, 3801-3809.
50. I. Kolouchová, O. Maf'átková, K. Sigler, J. Masák, and T. Řezanka, Folia Microbiol (Praha), 2016, 61, 431-438.
51. M. Kavšček, G. Bhutada, T. Madl, and K. Natter, BMC Syst. Biol., 2015, 9, 72.
52. M. Athenaki, C. Gardeli, P. Diamantopoulou, S. S. Tchakouteu, D. Sarris, A. Philippoussis, and S. Papanikolaou, J Appl. Microbiol., 2018, 124, 336-367.
53. C. Ratledge and J. P. Wynn, Adv Appl Microbiol, 2002, 51, 1-51.
54. R. Dulermo, H. Gamboa-Melendez, R. Ledesma-Amaro, F. Thévenieau, and J.-M. Nicaud, Biochim. Biophys. Acta, 2015, 1851, 1202-1217.
55. G. Singh, S. Sinha, K. K. Bandyopadhyay, M. Lawrence, and D. Paul, Microb. Cell Fact., 2018, 17, 182.
56. S. T. Coradetti, D. Pinel, G. M. Geiselman, M. Ito, S. J. Mondo, M. C. Reilly, Y.-F. Cheng, S. Bauer, I. V. Grigoriev, J. M. Gladden, B. A. Simmons, R. B. Brem, A. P. Arkin, and J. M. Skerker, Elife, 2018, 7.
57. S. Fillet, J. Gibert, B. Suárez, A. Lara, C. Ronchel, and J. L. Adrio, J. Ind. Microbiol. Biotechnol., 2015, 42, 1463-1472.
58. R. R. Bommareddy, W. Sabra, G. Maheshwari, and A.-P. Zeng, Microb. Cell Fact., 2015, 14, 36.
59. C. T. Walsh and T. A. Wencewicz, Nat Prod Rep, 2013, 30, 175-200.
60. T. Braunwald, L. Schwemmlein, S. Graeff-Hönninger, W. T. French, R. Hernandez, W. E. Holmes, and W. Claupein, Appl. Microbiol. Biotechnol., 2013, 97, 6581-6588.
61. M. I. Gonzalez-Siso, A. Garcïa-Leiro, N. Tarrío, and M. E. Cerdán, Microb. Cell Fact., 2009, 8, 46.
62. J. Xu, X. Zhao, W. Wang, W. Du, and D. Liu, Biochem EngJ, 2012, 65, 30-36.
63. L. J. Jonsson and C. Martin, Bioresour. Technol., 2016, 199, 103-112.
64. J. Shi, J. M. Gladden, N. Sathitsuksanoh, P. Kambam, L. Sandoval, D. Mitra, S. Zhang, A. George, S. W. Singer, B. A. Simmons, and S. Singh, Green Chem., 2013, 15, 2579.
65. F. Xu, D. Gage, and J. Zhan, J. Ind. Microbiol. Biotechnol., 2015, 42, 1149-1155.
66. E. D. Glowacki, G. Voss, and N. S. Sariciftci, Adv. Mater. Weinheim, 2013, 25, 6783-6800.
67. M. Irimia-Vladu, E. D. Glowacki, P. A. Troshin, G. Schwabegger, L. Leonat, D. K. Susarova, O. Krystal, M. Ullah, Y. Kanbur, M. A. Bodea, V. F. Razumov, H. Sitter, S. Bauer, and N. S. Sariciftci, Adv. Mater. Weinheim, 2012, 24, 375-380.
68. M. Sytnyk, E. D. Glowacki, S. Yakunin, G. Voss, W. Schöfberger, D. Kriegner, J. Stangl, R. Trotta, C. Gollner, S. Tollabimazraehno, G. Romanazzi, Z. Bozkurt, M. Havlicek, N. S. Sariciftci, and W. Heiss, J. Am. Chem. Soc., 2014, 136, 16522-16532.
69. T. A. Keating and C. T. Walsh, Curr. Opin. Chem. Biol., 1999, 3, 598-606.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A genetically modified fungal host cell capable of producing indigoidine, wherein the host cell comprises a non-ribosomal peptide synthetase (NRPS) that converts glutamine to indigoidine.

2. The genetically modified fungal host cell of claim 1, wherein the fungal host cell is a yeast host cell.

3. The genetically modified fungal host cell of claim 2, wherein the yeast host cell is a non-oleaginous yeast.

4. The genetically modified fungal host cell of claim 3, wherein the non-oleaginous yeast is a Saccharomyces species.

5. The genetically modified fungal host cell of claim 4, wherein the Saccharomyces species is Saccharomyces cerevisiae.

6. The genetically modified fungal host cell of claim 2, wherein the yeast host cell is an oleaginous yeast.

7. The genetically modified fungal host cell of claim 6, wherein the oleaginous yeast is a Rhodosporidium species.

8. The genetically modified fungal host cell of claim 7, wherein the Rhodosporidium species is Rhodosporidium toruloides.

9. The genetically modified fungal host cell of claim 1, wherein the NRPS is heterologous to the host cell.

10. The genetically modified fungal host cell of claim 9, wherein the NRPS is a bacterial NRPS.

11. The genetically modified fungal host cell of claim 10, wherein the NRPS is a Streptomyces lavendulae NRPS (BpsA).

12. The genetically modified fungal host cell of claim 1, wherein the NRPS comprises an amino acid sequence that is at least 70% identical to the amino acid sequence of SEQ ID NO:1, wherein the NRPS comprises the enzymatic activity to convert glutamine to indigoidine.

13. The genetically modified fungal host cell of claim 12, wherein the NRPS comprises a conserved domain, such as the amino acid sequence APRTETEKEI AEVWAKSLRR ESVSVQDDFF ESGGNSLIAV GLIRELNSRL GVSLPLQSVL ESPTVEKLSR RLEREV (SEQ ID NO:2), at the position corresponding to 937 to 1012 of SEQ ID NO:1.

14. The genetically modified fungal host cell of claim 12, wherein NRPS comprises a coiled coil structure, such as the amino acid sequence SRRLEREV (SEQ ID NO:3), or SRRLEREVAQESSRLVRLHAE (SEQ ID NO:4), at the position corresponding to 1005 to 1012, or 1005 to 1025, of SEQ ID NO:1, respectively.

15. The genetically modified fungal host cell of claim 1, wherein the serine at position 972 is a modified serine, such as 0-(pantetheine 4′-phosphoryl)serine.

16. A method for a genetically modified yeast host cell producing indigoidine, comprising (a) providing a genetically modified yeast host cell of the present invention, (b) culturing or growing the host cell in a suitable culture or medium such that indigoidine is produced, and (c) optionally extracting or separating the indigoidine from the rest of the culture or medium, and/or host cell.

17. The method of claim 16, wherein the providing step (a) comprises introducing a nucleic acid encoding the NRPS operatively linked to a promoter capable of expressing the NRPS in the host cell into the host cell.

18. The method of claim 16, wherein the culturing or growing step (b) comprises the host cell growing by respiratory cell growth.

19. The method of claim 16, wherein the culturing or growing step (b) takes place in a batch process or a fed-batch process, such as a high-gravity fed-batch process.

20. The method of claim 16, wherein the culture or medium comprises hydrolysates derived or obtained from a biomass, such as a lignocellulosic biomass.

21. The method of claim 16, wherein the culture or medium comprises one or more carbon sources, such as a sugar, such as glucose or galactose, or glycerol, or a mixture thereof.

22. The method of claim 21, wherein the carbon source is fermentable. In some embodiments, the carbon source is non-fermentable.

23. The method of claim 16, wherein the culture or medium comprises urea as a nitrogen course.

24. A method for constructing a genetically modified yeast host cell of the present invention, comprising (a) introducing a nucleic acid encoding the NRPS operatively linked to a promoter capable of expressing the NRPS in the host cell into the host cell.


SEQUENCE LISTING (4 sequences)

SEQ ID NO: 1 - protein, 1283 aa, Streptomyces lavendulae
MTLQETSVLEPTLRGTTTLPDLLAKRVAEHPEATAVAYRDEKLTYRELASRSSALAEYLRHLGVSTDDCVGLFVEPSIDLMVGAWGILSAGAAYLPLSPEYPEDRLRYMIENSQAKIILAQQRLVTRLRELAPQDVRVVTLRESEAFVLPEGQVAPAIEGARPDSLAYVIYTSGSTGKPKGVMIEHHSIVSQLGWLRETYGIDRSKTILQKTPMSFDAAQWEILSPANGATVVMGAPGVYADPEGLIETIVKYGVTTLQCVPTLLQGLLDTEKFPECTSLQQIFSGGEALSRLLAIQTTQEMPGRALINVYGPTECTINSSSYAVDPAELGEAPQSISIGAPVADTEYHILGKEDLKPVGVGEIGELYIGGGQLARGYLHRPDLTAERFLEIEVTEGAGPVRLYKTGDLGQWNPDGTVQFAGRADNQVKLRGYRVELDEISLAIENHDWVRNAAVIVKNDGRTGFQNLIACVELSEKEAALMDQGNHGSHHASKKSKLQVKAQLSNPGLRDDADLAARVAYDLPGAEPTPEQRSRVFARKTYRFYEGGAVTEADLLALLGGQVPAAYSRKAADLAPAELGQILRWFGQYLSEERLLPKYGYASPGALYATQLYFELEGVGGLQPGYYYYQPQRHQLVLISEKAATGRPTAHIHFIGKRGGIEPVYKNNIQEVLEIETGHIVGLFEQVLPAYGLDIRDLAYEPAVRDLLDVPEEDFYLGTFELVPHTGRREDHAEVYVQTHGSKVANLPEGQYRYADGTLTRFSDDIVLKKQVIAINQSVYQAASFGISVISRAPEEWMHYVTLGKKLQHLMMNGLGLGFMSSGYSSKTGNPLPASRRIDSVLQANGVESGPSYFFVGGRVSDEQLGHEGMREDSVHMRGPAELIRDDLVSFLPDYMIPNRVVVFERLPLSANGKIDAKALAASDQVNAELVERPFVAPRTETEKEIAEVWAKSLRRESVSVQDDFFESGGNSLIAVGLIRELNSRLGVSLPLQSVLESPTVEKLSRRLEREVAQESSRLVRLHAETGKDRPVLCWPGLGGYPMNLRTLAGEIGLGRSFYGIQAHGINEGEAPYATITEMAKADIEAIKELQPKGPYTLWGYSFGARVAFETAYQLEQAGEKVDNLFLIAPGSPTVRAENGKVYGREASFANRAYTTILFSVFTGTISGPDLEKCLESATDEESFAGFISELKGIDVDLAKRIISVVGQTYEFEYSFRELAERTLAAPVTIFKARGDDYSFIENSNGYSAEPPTVIDLDADHYSLLRTPDIGELVKHIRYLLGE

SEQ ID NO: 2 - protein, 76 aa, Streptomyces lavendulae
APRTETEKEIAEVWAKSLRRESVSVQDDFFESGGNSLIAVGLIRELNSRLGVSLPLQSVLESPTVEKLSRRLEREV

SEQ ID NO: 3 - protein, 8 aa, Streptomyces lavendulae
SRRLEREV

SEQ ID NO: 4 - protein, 21 aa, Streptomyces lavendulae
SRRLEREVAQESSRLVRLHAE